SEQUENCE LISTING (6 sequences)

SEQ ID NO: 1 - protein, 73 aa, Homo sapiens
MLGLVLALLSSSSAEEYVGLSANQCAVPAKDRVDCGYPHVTPKECNNRGCCFDSRIPGVPWCFKPLQEAECTF

SEQ ID NO: 2 - DNA, 398 bp, Homo sapiens
gatgctggggctggtcctggccttgctgtcctccagctctgctgaggagtacgtgggcctgtctgcaaaccagtgtgccgtgccggccaaggacagggtggactgcggctacccccatgtcacccccaaggagtgcaacaaccggggctgctgctttgactccaggatccctggagtgccttggtgtttcaagcccctgactaggaagacagaatgcaccttctgaggcacctccagctgcccctgggatgcaggctgagcacccttgcccggctgtgattgctgccaggcactgttcatctcagtttttctgtccctttgctcccggcaagctttctgctgaaagttcatatctggagcctgatgtcttaacgaataaaggtcccatgctccacccg

SEQ ID NO: 3 - protein, 41 aa, Homo sapiens
  MISC_FEATURE (1)..(1): Xaa = any amino acid
XCTVAPRERQNCGFPGVTPSQCANKGCCFDDTVRGVPWCFX

SEQ ID NO: 4 - protein, 42 aa, Homo sapiens
  MISC_FEATURE (1)..(1): Xaa = any amino acid
XCSRLSPHNRTNCGFPGITSDQCFDNGCCFDSSVTGVPWCFX

SEQ ID NO: 5 - protein, 41 aa, Homo sapiens
  MISC_FEATURE (1)..(1): Xaa = any amino acid
XCVMEVSDRRNCGYPGISPEECASRKCCFSNFIFEVPWCFX

SEQ ID NO: 6 - protein, 41 aa, Homo sapiens
  MISC_FEATURE (1)..(1): Xaa = any amino acid
XCAVPAKDRVDCGYPHVTPKECNNRGCCFDSRIPGVPWCFX

(19) United States
(12) Patent Application Publication
(10) Pub. No.: US 2006/0188471 A1
(43) Pub. Date: Aug. 24, 2006

(54) METHODS OF TREATING EPITHELIAL LESIONS

(76) Inventors: Daniel K. PODOLSKY, Wellesley, MA (US); Nicholas P. BARKER, Southborough, MA (US)

Correspondence Address: CLARK & ELBING LLP, 101 FEDERAL STREET, BOSTON, MA, 02110, US

(21) Appl. No.: 11/275,599

(22) Filed: Jan. 18, 2006

Related U.S. Application Data
(63) Continuation of application No. 10/698,572, filed on Oct. 31, 2003.
(60) Provisional application No. 60/422,708, filed on Oct. 31, 2002.

(51) Int. Cl. A61K 38/21; A61K 38/19; A61K 38/17; A61K 38/13; A61K 38/14; A61K 31/7072; A61K 31/7076; A61K 31/7034; A61K 31/496; A61K 31/4745; A61K 31/4704

(52) U.S. Cl. 424/85.1; 514/12; 514/165; 514/8; 424/85.4; 514/154; 514/11; 514/171; 514/43; 514/45; 514/35; 514/291; 514/200; 514/253.08; 514/312; 514/355; 514/570

(57) ABSTRACT

The invention features methods of preventing or treating epithelial cell lesions in a mammal by administering a composition containing a therapeutically effective amount of a trefoil domain-containing polypeptide, or a trefoil peptide fragment, and a mucoadhesive excipient. The invention further features methods of preventing or treating an eye disorder, e.g., dry eye, by topically administering to the eye a composition containing a therapeutically effective amount of a trefoil domain-containing polypeptide, or a trefoil peptide fragment, and a mucoadhesive excipient. Compositions containing a trefoil domain-containing polypeptide, or a trefoil peptide fragment, and a mucoadhesive excipient may be formulated in combination with one or more additional therapeutic agents and used in the methods of the invention.

This application is a continuation of U.S. application Ser. No. 10/698,572, filed Oct. 31, 2003, which claims benefit of U.S. Provisional Application No. 60/422,708, filed Oct. 31, 2002, each of which is hereby incorporated by reference.

### FIELD OF THE INVENTION

This invention related to the treatment of epithelial cell lesions.

### BACKGROUND OF THE INVENTION

Epithelial tissues line the surfaces of the body and internal organs and serve to protect them from the external environment. For instance, epithelial cells of the skin prevent or reduce desiccation and the harmful effects of UV radiation, aid in temperature homeostasis, and are the first line of defense to protect against chemical and thermal insults and infections by microbial pathogens. However, because these tissues cover the boundary between the external environment and internal organs, epithelial cells also provide a means to control the movement of nutrients and waste products into and out of the body. For example, epithelial tissue of the gastrointestinal tract (GI tract) protect the underlying tissue from the harmful effects of digestive enzymes, stomach acid, and ingested microorganisms. The epithelial cells also provide the absorptive surface for nutrient uptake.

Damage or infection to the epithelial cells may compromise the barrier and absorptive functions of the tissue. Thus, many epithelial cell populations have a high regenerative and restorative capacity. However, many pathological conditions and environmental insults can overwhelmed the reserve capacity of the epithelial tissue which have deleterious consequences to the organism as a whole. Thus, the maintenance of healthy epithelial tissue is of clear benefit for overall health and well being.

### SUMMARY OF THE INVENTION

The present invention features therapeutic methods and compositions for treating or preventing epithelial lesions in a mammal (e.g., a human) using trefoil domain-containing polypeptides (TDCPs) or trefoil peptide fragments (i.e., providing trefoil therapy) alone or in combination with other therapeutic agents. The TDCPs and trefoil peptide fragments of the invention preferably contain one or more trefoil domains having an amino acid sequence substantially identical to any one of SEQ ID NOs.: 3-6. Particularly useful trefoil peptide fragments include, for example, hITF₁₅₋₇₃, hITF25₋₆₂, hITF₂₂₋₆₂, hITF₂₁₋₆₂, hITF₂₅₋₇₀, hITF₂₂₋₇₀, hITF₂₅₋₇₂, hITF₂₂₋₇₂, hITF₂₁₋₇₂, hITF₂₅₋₇₃, hITF₂₂₋₇₃, and hITF₂₁₋₇₃. One particularly useful TDCP is EA-hITF₁₅₋₇₃.

The trefoil therapy of the present invention may be used in a method for treating or preventing epithelial lesions of the alimentary canal including the oral cavity, tongue, esophagus, stomach, small and large intestines and the distal bowel; the dermis or epidermis; the vaginal or cervical epithelia; the respiratory epithelia; or the corneal or conjunctival epithelium.

Lesions of the upper alimentary canal include, for example, mucositis and aphthous stomatitis, or may be caused by, for example, antineoplastic chemotherapy, chemical, thermal, or radiation bums, gingivitis, tooth decay, a biopsy procedure or surgical intervention, Behcet's disease, gastroesophageal reflux disease, and bacterial, viral, or fungal infections. Lesions of the distal bowel include, for example, mucositis, enteritis, proctitis, Crohn's disease, and ulcerative colitis, or may be caused by, antineoplastic chemotherapy, chemical, thermal, or radiation bums, a biopsy procedure, surgical intervention, tumor resection, and bacterial, viral, or fungal infections. Other lesions of the gastrointestinal tract include stomach ulcers.

Lesions of the dermis or epidermis include, for example, herpetic lesions, acne, a pressure ulcer, eczema, psoriasis, or may be caused by, for example, trauma, a surgical procedure, an allergen, chemical, thermal or radiation bums, and bacterial, viral, or fungal infections.

Lesions of the vaginal and cervical epithelia are caused by, for example, sexually transmitted diseases, antineoplastic therapy, trauma, an allergen, a biopsy, a surgical procedure, chemical, thermal or radiation bums, and bacterial, viral, or fungal infections.

Lesions of the respiratory epithelia are caused by, for example, an allergic reaction, asthma, chemical exposure, asbestos exposure, trauma, smoke or particulate matter inhalation, drug-induced lung damage, a surgical procedure, trauma from intubation, chronic pulmonary obstructive disease, hyperbaric oxygen therapy, and bacterial, fungal, or viral infections.

Disorders of the eye amenable to treatment using the methods and compositions of the present invention include, for example, superficial punctate keratitis, corneal ulcers, keratoconjunctivitis caused by herpes or an adenovirus infection, a traumatic physical injury, eye surgery, chemical exposure, UV light exposure, a keratoconus, a conjunctiva, a keratoconjunctivitis sicca (dry eyes), an ocular inflammation, a cicatricial penhigoid, a bacterial or a protozoal infection.

The TDCPs and trefoil peptide fragments of this invention may be formulated for administration by any route of that is appropriate for delivering a polypeptide therapeutic to the affected tissue. For example, TDCPs and trefoil peptide fragments may be formulated as an intravenous, intramuscular, subcutaneous, or intraocular injectable, or as an oral rinse, oral spray, ingestible liquid, suppository, enema, pessary, vaginal rinse, dry powder inhaler, nebulization solution, or eye drops.

The trefoil therapy may also be administered in conjunction with a second therapeutic agent. The second therapeutic agent may be administered in the same or different pharmaceutical composition and by the same or different route as the TDCP and/or trefoil peptide fragment. Also, the second therapeutic need not be administered with the same frequency or the same duration as trefoil therapy. Suitable second therapeutic agents include, for example, analgesics, anti-viral agents, antibacterial agents, anti-fungal agents, antiproliferative (i.e, chemotherapeutic) agents, anti-inflammatory agents, and steroids.

In a related aspect, the invention features isolated nucleic acid molecules and vectors containing the isolated nucleic acid molecules encoding trefoil domain-containing polypeptides (TDCPs) and trefoil peptide fragements, host cells (e.g., E. coli, P. pastoris, S. pombe, S. cerevisiae, Lactobacillus spp., and plant cells (e.g, monocots such as rice, wheat, corn, barley, and rye)) expressing those nucleic acids.

In another aspect, the invention features a method for producing recombinant hITF₂₁₋₇₂ or hITF₂₁₋₇₃ by providing a host cell such as a microorganism (e.g., bacteria and yeast) or a cultured human cell capable of expressing hITF₂₁₋₇₂ or hITF₂₁₋₇₃ and culturing that host cell at about pH 4.5-5.5. Preferably, the cell is cultured at about pH 5.0.

In another aspect, the invention features a method for producing recombinant hITF₁₅₋₇₂ or hITF₁₅₋₇₃ by providing a host cell such as a microorganism (e.g., bacteria and yeast) or a cultured human cell capable of expressing hITF₁₅₋₇₂ or hITF₁₅₋₇₃ and culturing that host cell at about pH 5.8-6.7. Preferably, the cell is cultured at about pH 6.0-6.5.

The TDCPs and trefoil peptide fragments of this invention may be monomeric, homodimeric, heterodimeric, or multimeric. Dimers can be either homodimers or heterodimers, and heterodimers may include one full length trefoil peptide (e.g., HITF) and any of the TDCPs or fragments of the invention, or any combination of TDCP and fragment. Desirably, both dimer members are biologically active. Trefoil domain-containing polypeptides and trefoil peptide fragments may be post-translationally modified, either by glycosylation, dipeptide addition, or proteolytic cleavage.

By “trefoil peptides” is meant all mammalian homologs of human spasmolytic polypeptide (hSP; also known as TFF2, GenBank Accession No. NM_{—}005423), human pS2 (also known as TFF1, GenBank Accession No. XM_{—}009779), human intestinal trefoil factor (hITF; also known as TFF3).

Mammalian trefoil peptides were discovered in 1982. One of the mammalian trefoil peptides, human intestinal trefoil factor (hITF; TFF3), has been characterized extensively, and is described in U.S. Pat. Nos. 6,063,755, and 6,221,840, hereby incorporated by reference. The other two known trefoil peptides are spasmolytic polypeptide (SP; TFF2) and pS2 (TFF1). Intestinal trefoil peptides, described extensively in the literature (e.g., Sands et al., Ann. Rev. Physiol. 58: 253-273, 1996), are expressed in the gastrointestinal tract and have a three-loop structure formed by intrachain disulfide bonds between conserved cysteine residues. These peptides protect the intestinal tract from injury and can be used to treat intestinal tract disorders such as peptic ulcers and inflammatory bowel disease. Homologs of these human polypeptides have been found in a number of non-human animal species. All members of this protein family, both human and non-human, are referred to herein as trefoil peptides.

The term “trefoil domain” is meant a polypeptide consisting of a sequence substantially identical to any of SEQ ID NOs:3-6, relating to the trefoil domains at hpS2₃₀₋₇₀, hSP1₃₀₋₇₁, hSP2₈₀₋₁₂₀, and hITF₂₄₋₆₄, respectively, and having the characteristic trefoil secondary structure. The aligned polypeptide sequences of the four identified human trefoil domains are shown in FIG. 1C. It is recognized in the art that one function of the six conserved cysteine residues is to impart the characteristic three-loop (trefoil) structure to the protein. The loop structure conforms to the general intrachain disulfide configuration of cys₁-cys₅ (corresponding to amino acid residues 25 and 51 of hITF; SEQ ID NO.:1), Cys₂-cys₄ (corresponding to amino acid residues 35 and 50 of hITF; SEQ ID NO.:1), and cys₃-cys₆ (corresponding to amino acid residues 45 and 62 of hITF; SEQ ID NO.:1).

By “fragment,” when referring to a trefoil peptide, is meant any polypeptide that, over the entire length of the fragment, is identical to a naturally occurring trefoil peptide and that contains a trefoil domain. Therefore, fragments of hITF/hTFF3 have 72 or fewer amino acids, fragments of hpS2/hTFF1 (GenBank Accession No. NP_{—}003216) 83 or fewer amino acids, and fragments of hSP/hTFF2 (GenBank Accession No. 1909187A) have 105 or fewer amino acids. In each case, the fragments may be significantly shorter than the full length protein. For example, the fragments may contains 65 or fewer, 58 or fewer, 52 or fewer, or as few as 42 amino acids. Particularly useful fragments of hITF/TFF3 include, for example, hITF₁₅₋₇₃, hITF₂₅₋₆₂, hITF₂₂₋₆₂, hITF₂₁₋₆₂, hITF₁₋₆₂, hITF₂₅₋₇₀, hITF₂₂₋₇₀, hITF₁₋₇₀, hITF₂₅₋₇₂, hITF₂₂₋₇₂, hITF₂₁₋₇₂, hITF₁₋₇₂, hITF₂₅₋₇₃, and hITF₂₂₋₇₃, (FIG. 1A).

By “trefoil domain-containing polypeptide” or “TDCP” is meant a polypeptide that contains a trefoil domain and, over its entire length, is not identical to a naturally occurring trefoil peptide. TDCPs may consist of a trefoil peptide fragment covalently bound to a second polypeptide or protein or a TDCP may consist of a polypeptide into which has been incorporated a trefoil domain. The second polypeptide or protein may impart other biological or therapeutic activities that are distinct from those normally attributed to a trefoil peptide. In the former case, the trefoil peptide fragment may covalently bound to the N-terminus, C-terminus, or have an internal linkage, such as a disulfide bond between a cysteine residue of the second polypeptide and, for example, an additional (seventh) cysteine residue of the fragment. In this configuration, it is preferable that the seventh cysteine is C-terminal to the trefoil domain. In the latter case, the trefoil domain may be incorporated into a “carrier” protein which may be a naturally occurring or an artificial polypeptide. The choice of carrier protein may be based on a desired biological or specific binding/targeting activity (or lack thereof) attributed to that protein. The carrier protein may, for example, alter the pharmacokinetic or pharmacodynamic profile of the combined molecule compared to the TDCP or fragment alone. For example, covalent attachment of a trefoil domain or trefoil peptide fragment to serum albumin may increase the serum half-life of the TDCP.

TDCPs and trefoil peptide fragments may exist as monomers, dimers, or multimers. TDCP or fragment monomers may form an interchain disulfide linkage to form a dimer, for example, ITF monomers may form an intrachain disulphide linkage at the cysteine residue that lies outside the trefoil domain. For example, the cysteine at position 71 of human ITF facilitates dimer formation.

TDCPs may be fragments of naturally occurring proteins (i.e., an endogenous mammalian protein) or be non-naturally occurring polypeptides. TDCPs that are non-naturally occurring do not have 100% amino acid sequence identity, over their entire length, to a naturally occurring protein. Preferably, however, a non-naturally occurring TDCP is substantially identical to a naturally occurring protein.

By “substantially identical,” when referring to a trefoil domain of SEQ ID NOs.:3-6, is meant an amino acid sequence which differs only by 4, 3, 2, or 1 amino acid insertions, substitutions, or deletions.

By “homodimer” is meant a TDCP or trefoil peptide fragment which forms an interchain covalent linkage (e.g., a disulfide linkage) with another TDCP of the same amino acid sequence to form a dimer. By “heterodimer” is meant a TDCP trefoil peptide fragment which forms an interchain covalent linkage with a TDCP or fragment of a different amino acid sequence or with any other trefoil peptide. For example, heterodimers may contain one ITF polypeptide and any TDCP. Alternatively, a TDCP heterodimer may have one TDCP disulfide-linked to a non-biologically active trefoil peptide or a non-trefoil peptide. Desirably, the interchain disulfide linkage will occur on a seventh cysteine residue of a trefoil peptide, for instance, corresponding to cys₇₁ of hITF; however, dimerization can be accomplished by any interchain disulfide linkage that does not disrupt biological activity. By a “multimer” is meant a TDCP or trefoil peptide fragment which forms one or more interchain disulfide linkage(s) with one or more TDCP and/or other polypeptides. Alternatively, a multimer may be formed through specific interactions in a non-covalent manner. For instance, an ITF homo- or heterodimer can recognize and specifically bind to mucopolysaccharides to form a multimer.

By a “multiple trefoil domain-containing polypeptide” or “MTDCP” is meant a non-naturally occurring polypeptide having two or more trefoil domains linked by peptide bonds, wherein the trefoil domains are separated by a linker sequence greater than 2 amino acids. MTDCPs are interchangeable with any TDCP or trefoil peptide fragment of the invention. Methods for the construction of chimeric MTDCPs are well established in the art using recombinant DNA technologies.

By “trefoil therapy” is meant a therapeutic composition or method containing or using one or more TDCP and/or trefoil peptide fragment of the invention.

By “post-translational modification” is meant a covalent, enzymatic, or chemical modification of a peptide, polypeptide, or protein. The post-translational modification may occur at a specific amino acid, or in the context of a discrete amino acid sequence resident in the peptide, polypeptide, or protein. Exemplary post-translational modifications include O-linked glycosylation occurring on serine residues, N-linked glycosylation occurring on asparagine residues, phosphorylation (O-linked on serine, threonine and tyrosine; N-linked on histidine, lysine and arginine), hydroxylation, sulfation, acetylation, acylation, and carboxylation. One of the most common post-translational modifications is proteolytic cleavage by specific and non-specific proteases as well as through non-enzymatic means, such as by chemical hydrolysis (for example, acid hydrolysis and CNBr-directed methionine cleavage). Specific proteases include the proprotein convertases (PCs) which act to cleave signal sequences such as the a-factor secretory sequence, serine proteases (e.g., chymotrypsin and trypsin), and methionine aminopeptidases. Other proteases include elastase, collagenase, aspartases, caspases, and the metalloproteases. Post-translational modifications also include the addition of amino acids or peptides to the native protein. For example, lysine residues may undergo ubiquitination or the amino terminal residue of a protein may undergo di-, tri- or polypeptide addition.

In desirable embodiment, TDCP or fragment is post-translationally modified to incorporate proteolytic processing,-dipeptide addition, and glycosylation. A peptide, polypeptide, or protein may encompass one or more post-translational modifications.

By “co-formulated” is meant any single pharmaceutical composition which contains two or more therapeutic or biologically active agents.

By “pharmaceutical composition” is meant any composition which contains at least one therapeutically or biologically active agent and is suitable for administration to a patient. For the purposes of this invention, pharmaceutical compositions suitable for delivering a therapeutic to the surface epithelia include, but are not limited to oral tablets and solutions, bio-erodable films, mucoadhesives, microspheres, creams, lotions, eye-drops, inhalants, suppositories, enemas, and pastes. Any of these formulations can be prepared by well known and accepted methods of art. See, for example, in Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York.

By “therapeutically effective amount” is meant an amount sufficient to provide medical benefit. When administering TDCPs or trefoil peptide fragments to a human patient according to the methods described herein, a therapeutically effective amount is usually about 1-2500 mg of active trefoil therapy per dose. Therapeutically effective amounts may range from as little as 10, 25, 50, 100, 200 mg per dose to as much as 300, 500, 750, 100, 1500, or 2000 mg per dose.

By “intestinal tract” is meant any portion of the digestive system distal to the pyloric sphincter, including the small and large intestines (encompassing the distal bowel).

By “distal bowel” is meant the portion of the digestive system that includes the ascending, transverse, descending, and sigmoid colon, rectum, and anal sphincter.

By “upper alimentary canal” is meant the portion of the digestive system proximal to the cardiac sphincter (cardioesophageal sphincter) of the stomach. Specifically, the upper alimentary canal is meant to include the oral cavity and associated structures (e.g., the tongue, gingival and sublingual tissues, and the hard and soft palates) and the esophagus.

The “alimentary canal” encompasses the upper alimentary canal, the stomach, intestinal tract, and ending at the anal sphincter.

By “biologically active,” when referring to a trefoil peptide, TDCP, or fragment is meant any polypeptide that exhibits an activity common to its related, naturally occurring family member, and that the activity is common to the family of naturally occurring intestinal trefoil peptides. In addition to the activities demonstrated herein, biological activities include, for example, the ability to alter gastrointestinal motility in a mammal, mucopolysaccaride binding, maintenance of the mucosa, and repair of mucosal integrity upon injury (see, for example, Taupin et al., Proc. Natl. Acad. Sci, USA, 97:799-804, 1999).

By “antimicrobial agent” is meant any compound that alters the growth of bacteria or fungal cells, protozoa, or viruses whereby growth is prevented, stabilized, or inhibited, or wherein the microbes are killed. In other words, the antimicrobial agents can be microbiocidal or microbiostatic.

By “antineoplastic therapy” is meant any treatment regimen used to treat cancer. Typical antineoplastic therapies include chemotherapy and radiation therapy.

### BRIEF DESCRIPTION OF THE DRAWINGS

FIGS. 1A-C are amino acid and nucleic acid sequences of trefoil peptides. FIGS. 1A and 1B are the amino acid sequence (Accession No. BAA95531; SEQ ID NO:1) and cDNA sequence (GenBank Accession No. NM_{—}003226; SEQ ID NO:2) of human intestinal trefoil factor respectively. FIG. 1C is a multisequence alignment of trefoil domains derived from human pS2/TFF1, SP/TFF2, and ITF/TFF3. X denotes any amino acid residue. Amino acids that share identity among the four trefoil domains are in bold.

FIG. 2 is an LC-MS chromatogram profile of a sample containing a purified recombinant hITF₁₅₋₇₃ standard.

FIG. 3 is an LC-MS chromatogram of a sample of fermentation broth from hITF₁₅₋₇₃-producing Pichia pastoris (run 16L-5) following methanol induction and grown in pH 5 culture broth at 30° C.

FIG. 4 is an LC-MS chromatogram of a sample of fermentation broth from hITF₁₅₋₇₃-producing Pichia pastoris (run 16L-5) following methanol induction and grown in pH 5 culture broth at 30° C. Samples were gently heated in the presence of dithiothreitol (DTT) for an additional 2 hours.

FIGS. 5A-5C are LC-MS chromatograms of a sample of fermentation broth from hITF₁₅₋₇₃-producing Pichia pastoris (run 16L-5) following methanol induction and grown in pH 5 culture broth at 30° C. and demonstrating the increase in complexity of the fermentation broth with respect to time of culturing. Samples were extracted at time points of T_{0 hrs} (FIG. 5A), T_{22 hrs} (FIG. 5B), and T_{1 hrs} (FIG. 5C).

FIG. 6 is an LC-MS chromatogram of a sample of fermentation broth taken at T_{0 hrs} of hITF₁₅₋₇₃-producing Pichia pastoris (run 16L-9) following methanol induction and grown in pH 6 culture broth at 30° C.

FIG. 7 is an LC-MS chromatogram of a sample of fermentation broth taken at T_{47.5 hrs} of hITF₁₅₋₇₃-producing Pichia pastoris (run 16L-9) following methanol induction and grown in pH 6 culture broth at 30° C.

FIG. 8 is an LC-MS chromatogram of a sample of fermentation broth taken at T_{72 hrs} of hITF₁₅₋₇₃-producing Pichia pastoris (run 16L-9) following methanol induction and grown in pH 6 culture broth at 30° C.

FIG. 9 is an LC-MS chromatogram of a sample of fermentation broth taken at T_{96 hrs} of hITF₁₅₋₇₃-producing Pichia pastoris (run 16L-9) following methanol induction and grown in pH 6 culture broth at 30° C.

FIGS. 10A and B are LC-MS chromatograms of a sample of fermentation broth from hITF₁₅₋₇₃-producing Pichia pastoris of the accumulating 18.5 minute eluate seen in FIGS. 6-9. FIG. 10A is the mass spec profile representing the mass/charge ratio and FIG. 10B shows the deconvoluted profile in atomic mass units.

FIG. 11 is a bar graph demonstrating the biological activity of ITF and polypeptide fragments of ITF on primary intestinal epithelial, IEC-6 cells. Motility assays were performed in Dulbecco's minimum eagle's media (DMEM) in 0.1% fetal bovine serum. Plate no.1 is treated with 20 ng/mL transforming growth factor-β, a known mitogen. Plate no. 2 shows the effect on IEC-6 cells in the presence of DMEM/0.1% FBS. Plate no. 3 shows the effect of recombinant purified hITF₁₅₋₇₃ at 100 μg/mL. Plate nos. 4-10 demonstrates the motility effect of hITF fragments (at 100 μg/mL) collected following hITF production in Pichia pastoris.

FIG. 12A are photomicrographs of confluent cultured IEC-6 cells treated as described in Example 4. The bottom panel shows the wound margin at t=0 hours. The top panel shows the wound margin following incubation with 1 mg/ml hITF₁₅₋₇₃ for 19 hours. FIG. 12B is a bar graph quantifying the number of IEC-6 cells that migrated across the wound margin during a 19 hour incubation with either 1 mg/ml bovine serum albumin (BSA; open bar) or 1 mg/ml hITF₁₅₋₇₃ (hITF; closed bar).

### DETAILED DESCRIPTION

The invention provides methods and compositions for the treating epithelial cell lesions. Lesions amenable to treatment using the methods and compositions of this invention include epithelial lesions of the dermis and epidermis (skin), alimentary canal including the epithelia of the oral cavity, esophagus, stomach, small and large intestines (anal sphincter, rectum, and colon, particularly the sigmoid colon and the descending colon), genitourinary tract (particularly the vaginal canal, cervix, and uterus), trachea, lungs, nasal cavity, and the eye. These lesions are treated by local application of a TDCP or a trefoil peptide fragment of the invention (i.e., by providing trefoil therapy), either alone or in combination with a second therapeutic agent and may be administered by any route that is useful for delivering therapeutics to appropriate target site.

Pharmaceutical Formulations

Alimentary Canal Compositions

Enemas

The enemas used to deliver the trefoil therapy of this invention are retention enemas, not evacuation enemas. Enemas, when administered in volumes of less than about 50 mL, deliver therapeutics to the rectum and sigmoid colon. However, enema volumes of about 150-250 mL can be used to deliver therapy to the descending, transverse and, in some cases, the ascending colon.

Trefoil-containing Enema for Treatment of Ulcerative Colitis

| | | |
|---|---|---|
| ITF₂₁₋₇₃ | 750 | mg |
| Sulfasalazine | 3 | grams |
| Distilled water | 250 | mL |

Bulk Enema Suspension Suitable for Refrigeration

| | | |
|---|---|---|
| ITF₂₁₋₇₃ | 5 | g/L |
| 5-aminosalicylic acid | 42 | g/L |
| NaH₂PO₄ | 0.4 | g/L |
| Na₂HPO₄ | 4.48 | g/L |
| NaCl | 9 | g/L |
| Sodium ascorbate | 0.5 | g/L |
| Tragacanth | 4 | g/L |
| Methylparaben | 2 | g/L |
| Propylparaben | 0.5 | g/L |
| Propylene glycol | 25 | mL/L |
| Distilled water | to 1 | L |

Suppositories

Suppositories are solid dosage forms for insertion into the rectum for delivering medication to the rectum and sigmoid colon. Typically, after insertion, the suppository softens, melts, disperses, or dissolves in the lumenal fluid. Rectal suppositories for adults are usually about 2-5 grams each and tapered on both ends. Infant suppositories are usually about half the size of the adult formulations.

Either a fatty or a water soluble/water miscible suppository base can be used in the compositions of this invention. Suitable fatty bases include, for example, cocoa butter, theobroma oil, vegetable oils modified by esterification, hydrogenation, glycerinated gelatin and high molecular weight polyethylene glycols. Sustained release and/or prolonged contact of the therapeutics can be achieved by proper selection of a fatty suppository base material. Cocoa butter, for example, melts quickly at body temperature but is immiscible with body fluids, resulting in a prolonged but low level delivery of fat-soluble therapeutics to the affected sites. Alternatively, water soluble or water miscible bases (e.g., polyethylene glycols and glycol-surfactant mixtures) typically dissolve or disperse quickly, resulting in a rapid delivery of the therapeutic to the affected sites.

An exemplary suppository formulation is provided below.

Trefoil-containing Suppository Tablet

| ITF₂₁₋₇₃ | 300 mg |
|---|---|
| Polyethylene glycol 1000 | 96% |
| Polyethylene glycol 4000 | 4% |

This formulation has a low-melting point and may require refrigeration to maintain in a solid state. Because the TDCPs and trefoil peptide fragments are proteinaceous, refrigeration may be desirable. The low melting point of the formulation results in rapid suppository melting following insertion, resulting in greater patient comfort. If refrigeration is not possible, or if heat molding techniques are to be used, the amount of polyethylene glycol 4000 may be increased to achieve a sufficiently heat stable formulation.

Other technical features of the trefoil-containing solutions are easily modified to suit the specific pharmaceutical formulation and the clinical indication being treated. For example, the pH and osmolarity of the formulation may be adjusted to confer trefoil peptide stability, while minimizing gastrointestinal irritancy and sensitivity.

Oral Sprays, Rinses, and Emulsions

Spray systems are particularly useful for delivering therapeutics to the upper alimentary canal and respiratory tract. Suitable spray delivery systems include both pressurized and non-pressurized (pump actuated) delivery devices. The trefoil-containing solution, delivered as an oral spray, is preferably an aqueous solution; however, organic and inorganic components, emulsifiers, excipients, and agents that enhance the organoleptic properties (i.e., flavoring agents or odorants) may be included. Optionally, the solution may contain a preservative that prevents microbial growth (i.e., methyl paraben). Although water itself may make up the entire carrier, typical liquid spray formulations contain a co-solvent, for example, propylene glycol, corn syrup, glycerin, sorbitol solution, surfactants and the like, to assist solubilization and incorporation of water-insoluble ingredients. In general, therefore, the compositions of this invention preferably contain from about 1-95% v/v and, most preferably, about 5-50% v/v, of the co-solvent. When prepared as a spray, patients typically self-administer 1-5 times per day. The spray delivery system is normally designed to deliver 50-100 μl per actuation, and therapy may require 1-5 actuations per dose. The Theological properties of the spray formulation are optimized to allow shear and atomization for droplet formation. Additionally, the spray delivery device is designed to create a droplet size which promotes retention on mucosal surfaces of the upper alimentary canal or respiratory tract.

Compositions suitable for oral sprays or the alimentary canal can also be formulated as an oral rinse or mouthwash. Administration of TDCPs and trefoil peptide fragments using these formulations is typically done by swishing, gargling, or rinsing the oral cavity with the formulation. Optionally, these formulations can be swallowed, providing trefoil peptide therapy to the esophagus, stomach, and/or intestines. This delivery method is particularly useful for treating patients suffering related disorders of the intestinal epithelium. For example, patients receiving antineoplastic chemotherapy, in addition to oral mucositis, frequently develop more distal lesions of the gastrointestinal tract such as lesions of the gastric and intestinal epithelium. It is well known that intestinal trefoil peptides, particularly hITF, are stable at stomach pH. Thus, swallowing a trefoil-containing solution designed primarily for treating oral mucositis may also benefit lesions of the lower alimentary canal (i.e., stomach and intestines).

Other technical features of the trefoil-containing solutions are easily modified to suit the specific pharmaceutical formulation and the clinical indication being treated. For example, the pH and osmolality of the formulation may be adjusted to confer trefoil peptide stability, while minimizing oral irritancy and sensitivity.

Ointments, Pastes, and Gels

Lesions of the oral and esophageal epithelium caused by trauma are amenable to TDCP therapy delivered as an ointment, paste, or gel. The viscous nature of these types of preparations allows for direct application into the wound site. Optionally, the wound site can be covered with a dressing to retain the trefoil-containing composition, protect the lesion from trauma, and/or absorb exudate. As discussed further below, these preparations are particularly useful to restore epithelial integrity following traumatic surgical procedures such as, for example, tooth extraction, tissue biopsy, or a tumor resection. Such viscous formulations may also have a local barrier effect thereby reducing irritation and pain.

Chewable Tablets, Lozenges, and Conifectionaries

Preparing a trefoil-containing composition as a chewable tablet, lozenge, or a confectionary such as chewing gum provides several advantages to traditional drug delivery vehicles. First, prolonged contact and sustained release at the target site (mouth and esophagus) is achieved. Second, such formulations often results in higher patient compliance, especially when administering trefoil peptides to children.

Formulations for chewable tablets are well known and typically contain a base of sugar, starch, or lipid and a flavoring agent. An exemplary formulation for a chewable tablet is provided below.
Trefoil-containing Chewable Tablet Formulation (per tablet)
ITF₂₁₋₇₃—300 mg
Mannitol—675 mg
Microcrystalline cellulose—75 mg
Corn starch—30 mg
Calcium sterate—22 mg

The incorporation of therapeutics into chewing gum and other confectionary style formulations is known in the art (e.g., U.S. Pat. No. 5,858,391). Any chewable or oral retentive formulation may also include a flavoring agent, for example, sodium saccharin or peppermint oil.

Genitourinary Compositions

Vaginal Rinses

A vaginal rinse, or douche, is used to deliver the intestinal trefoil peptides to the cells of the vagina and cervix. Douche volumes of about 50-300 mL can be used.

Trefoil-containing Douche for Treating Minor Vaginal Irritation

| | |
|---|---|
| ITF₂₁₋₇₃ | 500 mg |
| Povidone-iodine | 0.30% |
| Distilled water | 150 mL |

Vaginal Suppositories and Pessaries

Suppositories are solid dosage forms for insertion into the vagina for delivering medication to the vagina, cervix, and uterus. Typically, after insertion, the suppository softens, melts, disperses, or dissolves. Vaginal suppositories are usually about 1-7 grams each and tapered on both ends. Either a fatty or a water soluble/water miscible suppository base can be used in the compositions of this invention. Suitable fatty bases include, for example, cocoa butter, starch, theobroma oil, vegetable oils modified by esterification, hydrogenation, glycerinated gelatin, and high molecular weight polyethylene glycols. Sustained release and/or prolonged contact of the therapeutics can be achieved by proper selection of a fatty suppository base material. Cocoa butter, for example, melts quickly at body temperature but is immiscible with body fluids, resulting in a prolonged but low level delivery of fat-soluble therapeutics to the affected sites. The preparation and use of vaginal suppositories and pessiaries are well known in the art. Many of the formulations are similar to those of rectal suppositories.

Vaginal Ointments, Pastes, and Gels

Lesions of the vaginal and cervical epithelium and of the external genitalia and the surrounding skin are amenable to trefoil therapy delivered as an ointment, paste, or gel. The viscous nature of these types of preparations allows for direct application into the wound site. Optionally, the wound site can be covered with a dressing to retain the trefoil-containing composition, protect the lesion and/or absorb exudate. As discussed further below, these preparations are particularly useful to restore epithelial integrity following traumatic surgical procedures (e.g., episiotomy). Such viscous formulations may also have a local barrier effect thereby reducing irritation and pain. Alternatively, the ointment, paste, or gel composition may contain, in addition to a TDCP or a trefoil peptide fragment, an antimicrobial such as an antifungal agent. These combinations are particularly useful for treating vaginal infections and certain sexually transmitted diseases.

Trefoil-containing Paste for Treating Candidosis

| | | |
|---|---|---|
| ITF₂₁₋₇₃ | 500 | mg |
| Tioconazole | 300 | mg (6.5%) |
| White Petrolatum | 4.6 | grams |

Mucoadhesives

A mucoadhesive excipient can be added to any of the previously described pharmaceutical compositions. The mucoadhesive formulations coat the lesioned area, resulting in retention of the intestinal trefoil peptide at the lesion site, providing protection, inhibiting irritation, and accelerating healing of inflamed or damaged tissue. Mucoadhesive formulations suitable for use in these pharmaceutical preparations are well known in the art (e.g., U.S. Pat. No. 5,458,879). Particularly useful mucoadhesives are hydrogels composed of about 0.05-20% of a water-soluble polymer such as, for example, poly(ethylene oxide), poly(ethylene glycol), poly(vinyl alcohol), poly(vinyl pyrrolidine), poly(acrylic acid), poly(hydroxy ethyl methacrylate), hydroxyethyl ethyl cellulose, hydroxy ethyl cellulose, chitosan, and mixtures thereof. These polymeric formulations can also contain a dispersant such as sodium carboxymethyl cellulose (0.5-5.0%).

Other preferred mucoadhesive excipients for liquid compositions are ones that allow the composition to be administered as a flowable liquid but will cause the composition to gel in the lumenal milieu or upon contact with extracellular fluids or secretions, thereby providing a bioadhesive effect which acts to hold the therapeutic agents at the lesion site for an extended period of time. The anionic polysaccharides pectin and gellan are examples of materials which when formulated into a suitable composition will gel in the lumenal fluid. These types of mucoadhesive preparations are particularly useful in the alimentary canal, especially the upper alimentary canal and distal bowel because of the relatively high concentration of cations. The liquid compositions containing pectin or gellan will typically consist of 0.01-20% w/v of the pectin or gellan in water or an aqueous buffer system.

Other useful compositions which promote mucoadhesion and prolonged therapeutic retention in surface epithelia are colloidal dispersions containing 2-50% colloidal particles such as silica or titanium dioxide. Such formulations form as a flowable liquid with low viscosity suitable as an enema; however, the particles interact with glycoprotein, especially mucin, transforming the liquid into a viscous gel, providing effective mucoadhesion (e.g., U.S. Pat. Nos. 5,993,846 and 6,319,513).

In an alternatives formulation, the TDCP and/or other therapeutics can be encapsulated in bioerodible microspheres rather than being dissolved in the aqueous phase of the formulation. A wide variety of microencapsulation drug delivery systems have been developed and many share similar polymeric compositions as used for bioerodible films (described below). Polymers commonly used in the formation of microspheres include, for example, poly-ε-caprolactone, poly(ε-caprolactone-Co-DL-lactic acid), poly(DL-lactic acid), poly(DL-lactic acid-Co-glycolic acid) and poly(ε-caprolactone-Co-glycolic acid) (see, for example, Pitt et al., J. Pharm. Sci., 68:1534, 1979; Davis et al. Microsphere and Drug Therapy, Elsevier, 1984; Benoit et al. Biodegradable Microspheres: Advances in Production Technologies, Chapter 3, Ed. Benita, S, Dekker, New York, 1996; Microencapsulation and Related Drug Processes, Ed. Deasy, Dekker, 1984, New York; U.S. Pat. No. 6,365,187). Preferably, the microspheres are bioadhesive or are prepared in formulations containing a bioadhesive excipient.

Bioerodible Film Delivery Devices

Polymeric film devices provide several advantages for therapeutic delivery to the oral cavity. Unlike rinses, pastes, gels, and other flowable compositions, a film device can reside for prolonged periods of time (i.e., hours to days) in the oral cavity and provide sustained release throughout its residency. Typically, the film is partially or completely bioerodible and contains a mucoadhesive layer to fasten the film to the oral mucosa. Film devices, in addition to its use for delivering therapeutics, can also provide protection against mechanical injury or microbial infection of a lesion site. This physical barrier function is particularly advantageous when treating conditions such as mucositis or aphthous stomatitis. Additionally, as discussed further below, a film device can be used to release the TDCP directly onto the underlying mucosa, into the lumen of the oral cavity, or a combination of both.

Film devices consist of at least two layers; a mucoadhesive layer suitable for attaching the film to the oral mucosa and a bulk layer which contains the active therapeutic(s). Many suitable mucoadhesives are known in the art and are discussed above. Optionally, one or more therapeutics can also be provided in the adhesive layer.

The bulk layer of the composite delivery device may be made of one or more bioerodible polymeric materials. Suitable polymers include, for example, starch, gelatin, polyethylene glycol, polypropylene glycol, polyethylene oxide, copolymers of ethylene oxide and propylene oxide, copolymers of polyethylene glycol and polypropylene glycol, polytetramethylene glycol, polyether urethane, hydroxyethyl cellulose, ethyl cellulose, hydroxypropyl cellulose, hydroxypropylmethyl cellulose, alginate, collagen, polylactide, poly(lactide-co-glycolide) (PLGA), calcium polycarbophil, polyethymethacrylate, cellulose acetate, propylene glycol, polyacrylic acid, crosslinked polyacrylic acid, hydroxyethyl methacrylate/methyl methacrylate copolymer, silicon/ethyl cellulose/polyethylene glycol, urethane polyacrylate, polystyrene, polysulfone, polycarbonate, polyorthoesters, polyanhydrides, poly(amino acids), partially and completely hydrolyzed alkylene-vinyl acetate copolymers, polyvinyl chloride, polymers of polyvinyl acetate, polyvinyl alkyl ethers, styrene acrylonitrile copolymers, poly(ethylene terphthalate), polyalkylenes, poly(vinyl imidazole), polyesters and combinations of two or more of these polymers.

A particularly useful bulk layer polymer consists of PLGA and ethyl cellulose. PLGA is bioerodible and can be formulated to degrade over a wide range of conditions and rates. Ethyl cellulose is a water-insoluble polymer that can act as a plasticiser for the PLGA when a film is formed, but will be eroded in a bodily fluid. Due to its water-insolubility, it also has an effect on the degree and rate of swelling of the resultant film.

An optional third layer which is impermeable to the TDCP/fragment can also be added to the wafer. Preferably, this barrier layer is also bioerodible. Suitable barrier layer polymers include ethyl cellulose, poly(acrylic acid), or other polyelectrolytes. In one configuration, the barrier layer is placed on the opposite side of the bulk layer relative to the adhesive layer, thereby directing the released therapeutic agent onto the contacted epithelium rather than being diluted in the lumenal fluid. This configuration is particularly useful for treating discrete lesions (i.e., mucositis or aphthous stomatitis) of the tongue, sublingual tissue, or buccal mucosa. In an alternative configuration of the film device, the barrier layer is placed between the bulk layer and the adhesive layer. This configuration directs therapeutic release into the lumen of the oral cavity and is useful for treating more diffuse lesions of the tongue, oral cavity, and esophagus. The configuration is also useful for delivering therapeutics which are cytotoxic when administered at high concentrations because it has the effect of shielding the underlying tissue from direct contact with the therapeutic-containing film.

Dermal Formulations: Ointments, Pastes, Creams, Gels, Irrigation Solutions, and Tissue Adhesives

Lesions of the epithelium of the skin, such as those resulting from trauma or inflammation, are amenable trefoil therapy administered as an ointment, paste, or gel. The viscous nature of these types of preparations allows for direct application into the wound site. Optionally, the wound site can be covered with a dressing to retain the trefoil-containing composition, protect the lesion and/or absorb exudate. As discussed further below, these preparations are particularly useful to restore epithelial integrity following traumatic surgical procedures (e.g., skin biopsies). Such viscous formulations may also have a local barrier effect thereby reducing irritation and pain. Further, mucoadhesive agents, as previously described, may be incorporated into the ointments, creams, pastes, gels, and oils of this invention. The mucoadhesives prolong the retention of the trefoil-containing formulation at the lesion site.

Bioadhesives and bioerodible polymers are useful as an alternative method of wound closure, and may be used as drug delivery vehicles. Bioadhesives are a particularly useful alternative to sutures, for wound closure in geriatric populations, where the skin is particularly friable. Any of the well-known bioadhesives or polymers is suitable for use with the TDCPs and trefoil peptide fragments of this invention (e.g. U.S. Pat. Nos. 5,990,194, 6,159,498, and 6,284,235). The TDCPs and fragments are incorporated into the adhesive or polymer by any method suitable for incorporating any other therapeutic agent into these products. The particular method will depend on the chemical composition of the product and the manufacturing process.

In addition, TDCPs and fragments can also be present in any of the known irrigation solutions (e.g. 0.9% saline or Ringer's solution) for surgical purposes.

Suture Materials and Wound Dressings

Suture materials, sterile wound dressings, bioerodible polymers and tissue adhesives can be impregnated with the TDCPs and trefoil peptide fragments of the present invention and used at an incision site to promote dermal and epidermal healing.

These formulations can be made according to known and conventional methods for preparing such formulations. For example, sutures made from monofilaments can be impregnated by loading the polymer solution with a TDCP or fragment prior to extrusion. Suture material can also be impregnated by repeated soaking/drying cycles using a trefoil-containing solution. The number of cycles depends on the concentration of TDCP/fragment in the soaking solution and the final amount to be contained in the suture. Soaking is a particularly effective impregnation method for braided suture materials because the TDCP/fragment is retained by the surface contours.

Sterile dressings and gauzes for wounds and bums, impregnated with a TDCP or trefoil peptide fragment, can also be prepared by standard methods. Typically, the TDCP/fragment will be present in a viscous gel (e.g., hydrogel), separated from the dermal lesion by a permeable fabric that does not adhere to the wound.

Treatment of Eye Disorders

Injury to the corneal epithelium results in the rapid formation of a layer of cells that covers the denuded corneal surface, preventing infection and loss of vision. After wounding, resealing of the surface epithelium occurs over a period of several hours, resulting in the formation of a migratory leading edge. Proliferation through mitotic burst is observed in cells surrounding the original wound margin after 36 hours.

The invention features a method for treating an eye disorder (trauma or lesion) in a patient by administering trefoil therapy. An eye disorder may affect any part of the eye, e.g., the cornea, the sclera, the retina, the conjunctiva, the ciliary body, the posterior chamber, or the anterior chamber. In a preferred embodiment the eye disorder affects the cornea, e.g., the corneal epithelium, or the conjunctiva. Eye disorders include but are not limited to superficial puntate keratitis, corneal ulcer, herpes simplex keratoconjunctivitis, ophthalmic herpes zoster, phlyctenular keratoconjunctivitis, keratoconus, conjunctiva, keratoconjunctivitis sicca (dry eyes), ocular inflammation, corneal ulcers and cicatricial penhigoid. Eye disorders can be caused by viruses (e.g., adenoviruses, herpes simplex virus), blepharitis, keratitis sicca, trachoma, corneal foreign bodies, ultraviolet light exposure (e.g., welding arcs, sunlamps), contact lens overwear, systemic drugs (e.g., adenine arabinoside), tropical drugs, bacteria, protozoa, fungi, or by a hypersensensitive reaction to a known or unknown antigen.

Physical eye trauma can also result in an eye disorder. Physical trauma to the eye includes an abrasion to the cornea (e.g., caused by a foreign body), perforation of the cornea (e.g., caused by a blunt injury that disrupts the continuity of the cornea), or chemical burns to the cornea (e.g., exposure to NaOH), or through surgical procedures (e.g., corneal transplants and intraocular injections). The eye disorder generally results in damage and disruption of eye function or structure. For example, the disorder may cause the corneal epithelium to tear, cause necrosis of the cornea, cause corneal ulcers or damage the conjunctiva. Any of the eye disorders listed above can be treated with trefoil therapy.

Therapeutics Agents

Trefoil Domain-Containing Polypeptides and Trefoil Peptide Fragments

The TDCPs and trefoil peptide fragments are administered at 1-5000 mg per dose, preferably 5-2500 mg per dose, or more preferably 10-1500 mg per dose, depending on the nature and condition of the lesion being treated, the anticipated frequency and duration of therapy, the route of administration, and the type of pharmaceutical composition used to deliver the trefoil therapy. Trefoil therapy is typically administered 1-5 times per day.

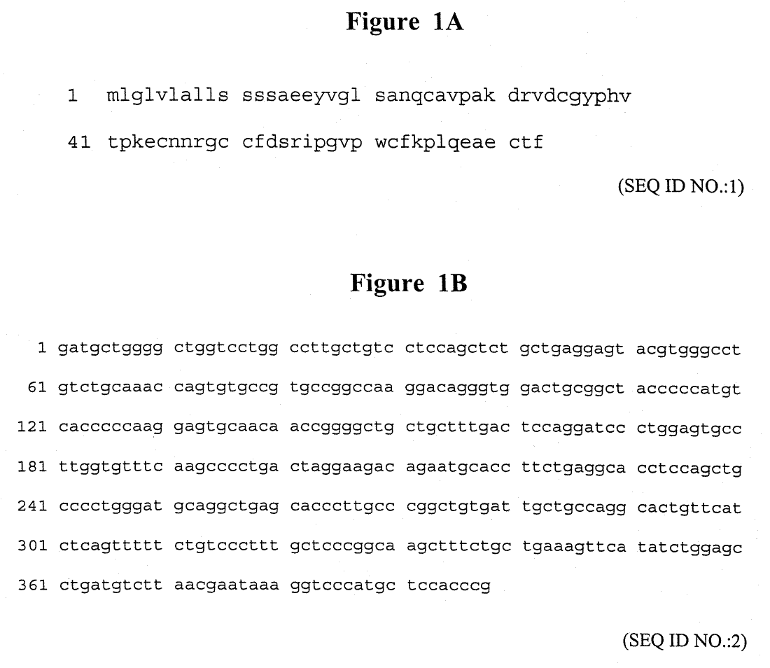

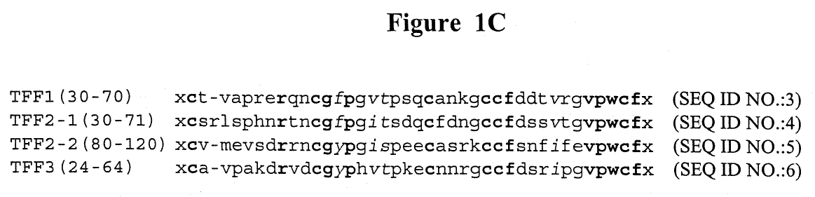

Particularly useful TDCPs and trefoil peptide fragments that retain biological activity include the polypeptide corresponding to amino acid residues 15-73 (hITF₁₅₋₇₃) of FIG. 1A. Other useful fragments include hITF₂₁₋₇₃, hITF₂₂₋₇₃, and hITF₂₅₋₇₃. Biologically active ITF polypeptide fragments are formed following cleavage of the C-terminal phenylalanine residue (i.e., hITF₁₋₇₂, hITF₁₅₋₇₂, hITF₂₁₋₇₂, and hITF₂₂₋₇₂, and hITF₂₅₋₇₂), following cleavage or termination at the penultimate cysteine residue (i.e. hITF₁₋₆₂, hITF₂₁₋₆₂, and hITF₂₂₋₆₂, and hITF₂₅₋₆₂), following cleavage or termination before the final cysteine residue (i.e., hITF₁₋₇₀, hITF₁₅₋₇₀, hITF₂₁₋₇₀, and hITF₂₂₋₇₀, and hITF₂₅₋₇₀).

The TDCPs and fragments of this invention can be produced using any appropriate method. For example, cDNA encoding the desired TDCP can be used with any method known in the art for producing recombinant proteins. Exemplary methods are provided herein. All TDCPs and trefoil peptide fragments, particularly hITF₁₅₋₇₃ and hITF₂₁₋₇₃ can be produced using a Pichia yeast expression system (see, for example, U.S. Pat. Nos. 4,882,279 and 5,122,465) transformed with a cDNA encoding trefoil peptide fragment species, such as the full length hITF or ITF₂₁₋₇₃, when the fermentation culture is maintained at pH ˜5.0.

Antiproliferative Agents

Particularly useful antiproliferative agents that can be administered in the combinations of the invention are microtubule inhibitors, topoisomerase inhibitors, platins, alkylating agents, and anti-metabolites. Exemplary antiproliferative agents include paclitaxel, gemcitabine, doxorubicin, vinblastine, etoposide, 5-fluorouracil, carboplatin, altretamine, aminoglutethimide, amsacrine, anastrozole, azacitidine, bleomycin, busulfan, carmustine, chlorambucil, 2-chlorodeoxyadenosine, cisplatin, colchicine, cyclophosphamide, cytarabine, cytoxan, dacarbazine, dactinomycin, daunorubicin, docetaxel, estramustine phosphate, floxuridine, fludarabine, gentuzumab, hexamethylmelamine, hydroxyurea, ifosfamide, imatinib, interferon, irinotecan, lomustine, mechlorethamine, melphalen, 6-mercaptopurine, methotrexate, mitomycin, mitotane, mitoxantrone, pentostatin, procarbazine, rituximab, streptozocin, tamoxifen, temozolomide, teniposide, 6-thioguanine, topotecan, trastuzumab, vincristine, vindesine, and vinorelbine.

The combinations of compounds of the invention are useful for the treatment of neoplasms. Combination therapy may be performed alone or in conjunction with another therapy (e.g., surgery, radiation, chemotherapy, biologic therapy). Additionally, a person having a greater risk of developing a neoplasm (e.g., one who is genetically predisposed or one who previously had a neoplasm) may receive prophylactic treatment to inhibit or delay neoplastic formation. The duration of the combination therapy depends on the type of disease or disorder being treated, the age and condition of the patient, the stage and type of the patient's disease, and how the patient responds to the treatment.

The dosage, frequency and mode of administration of each component of the combination can be controlled independently. For example, one compound (i.e., the pyridinone analog) may be administered topically three times per day, while the second compound (i.e., the antiproliferative) may be administered orally once per day. Combination therapy may be given in on-and-off cycles that include rest periods so that the patient's body has a chance to recovery from any as yet unforeseen side-effects. The compounds may also be formulated together such that one administration delivers both compounds.

Anti-Inflammatory Agents

Any suitable anti-inflammatory agent can be formulated with the trefoil peptide and employed using the method of this invention. Suitable anti-inflammatory agents include, but are not limited to non-steroidal anti-inflammatory drugs (e.g., salicylic acid derivatives, indomethacin, ibuprofen, tacrolimus, acetaminophen), cyclooxygenase-2-specific inhibitors such as rofecoxib (Vioxx®), celecoxib (Celebrex®, etodolac, and nimesulide), topical glucocorticoid agents and specific cytokines directed at T lymphocyte function. Anti-inflammatory concentrations known to be effective following rectal administration can be used. For example, ibuprofen may be present in the composition at concentrations sufficient to deliver between 25-800 mg per day to the lesion. Corticosteroids may be co-formulated with a trefoil peptide at concentrations known to be effective for local rectal use.

Antimicrobial Agents

Any of the many known antimicrobial agents can be used in the compositions of the invention at concentrations generally used for these agents. Antimicrobial agents include antibacterials, antifungals, antiprotozoal, and antivirals.

Examples of antibacterial agents (antibiotics) include the penicillins (e.g., penicillin G, ampicillin, methicillin, oxacillin, and amoxicillin), bacitracin, the cephalosporins (e.g., cefadroxil, ceforanid, cefotaxime, and ceftriaxone), the tetracyclines (e.g., doxycycline, minocycline, and tetracycline), the aminoglycosides (e.g., amikacin, gentamycin, kanamycin, neomycin, streptomycin, and tobramycin), the macrolides (e.g., azithromycin, clarithromycin, and erythromycin), the fluoroquinolones (e.g., ciprofloxacin, lomefloxacin, and norfloxacin), and other antibiotics including chloramphenicol, clindamycin, cycloserine, isoniazid, rifampin, and vancomycin.

Antiviral agents are substances capable of destroying or suppressing the replication of viruses. Examples of anti-viral agents include 1,-D-ribofuranosyl-1,2,4-triazole-3 carboxamide, 9-2-hydroxy-ethoxy methylguanine, adamantanamine, 5-iodo-2′-deoxyuridine, trifluorothymidine, interferon, adenine arabinoside, protease inhibitors, thymidine kinase inhibitors, sugar or glycoprotein synthesis inhibitors, structural protein synthesis inhibitors, attachment and adsorption inhibitors, and nucleoside analogues such as acyclovir, penciclovir, valacyclovir, and ganciclovir.

Antifungal agents include both fungicidal and fungistatic agents such as, for example, Amphotericin B, butylparaben, clindamycin, econaxole, fluconazole, flucytosine, griseofulvin, nystatin, ciclopirox, and ketoconazole.

Antiprotozoal agents are directed to treating protozoan infections. Examples of antiprotozoal chemotherapeutics are the amebicides (diloxanide furoate, iodoquinol, paromomycin, dehydroemetine, metronidizole, tinidizole and ornidazole) used to treat amebiasis, giardiasis, and trichomoniasis.

Analgesics and Anesthetics

Any of the commonly used topical analgesics can be used in the compositions of the invention. The analgesic is present in an amount such that there is provided to the distal bowel lesion a concentration of between one-half and five percent concentration for lidocaine (5-50 mg/mL in 20-40 mL per dose of liquid). Examples of other useful anesthetics include procaine, lidocaine, tetracaine, dibucaine, benzocaine, p-buthylaminobenzoic acid 2-(diethylamino) ethyl ester HCl, mepivacaine, piperocaine, and dyclonine.

Other analgesics include opioids such as, for example, morphine, codeine, hydrocodone, demorol, and oxycodone. Any of these analgesics may also be co-formulated with other compounds having analgesic or anti-inflammatory properties, such as acetaminophen, aspirin, and ibuprofen.

Steroids

Steroids may be used to treat lesions of the distal bowel. For example, ulcerative colitis may be treated using a paste preparation of triamcinolone (0.1%), hydrocortisone, fluticasone, budesonide, or beclomethasone.

5-Aminosalicylate Derivatives

5-aminosalicylate (5-ASA) derivatives are known to be useful for treating inflammatory bowel diseases such as Crohn's Disease and ulcerative colitis. Particularly useful 5-ASA derivatives include, for example, sulfasalazine, mesalamine, olsalazine, and balsalazide. Sulfasalazine is typically administered as a 3% enema, or orally in doses of 500-1000 mg. Mesalamine is normally administered as a one gram enema, daily for 3-6 weeks, or as a 500 mg suppository, 2-3 times per day for 3-6 weeks. Similar formulations may be prepared for any 5-ASA derivatives.

Opthalinological Antimicrobial Agents

Infectious diseases of the eyes are encountered regularly in clinical practice. Examples of eye infections and modes of antimicrobial chemotherapeutics are given below. Dacryocystitis (an infection to the lacrimal sac), hordeolum and blephartis (infections to the eyelids), conjunctivitis and keratitis are most commonly caused by infections by microbial pathogens, for example, herpespes virus, herpes zoster virus, adenovirus, Staphylococcus aureus, Streptococccus pneumoniae, Haemophilus and Neisseria spp. Topical ophthalmologic-grade antibiotic and antifungal solutions and ointments are prepared, typically between 0.1-5% (w/v) in solution for treating bacterial pathogens. Antiviral solutions of idoxuridine, trifluridine, and vidarabine ointments or solutions are prepared at 0.1-1% w/v, while foscarnet, acyclovir, gancyclovir, formivirsen, and cidoforvir are administered orally, intravenously, topically to the eye, or through intravitral implant.

Production of TDCPs and Trefoil Peptide Fragments

TDCPs and trefoil peptide fragments can be produced by any method known in the art for expression of recombinant proteins. Nucleic acids that encode the desired polypeptide may be introduced into various cell types or cell-free systems for expression thereby allowing small-, large-, and commercial-scale production, purification, and patient therapy.

Eukaryotic and prokaryotic expression systems may be generated in which a TDCP or trefoil peptide fragment-coding sequence is introduced into a plasmid or other vector, which is then used to transform living cells. Constructs in which the trefoil peptide cDNA contains the entire open reading frame or biologically active fragment thereof, are inserted in the correct orientation into an expression plasmid and may be used for protein expression. Prokaryotic and eukaryotic expression systems allow for the expression and recovery of fusion proteins in which the TDCP/fragment is covalently linked to a tag molecule on either the amino terminal or carboxy terminal side, which facilitates identification and/or purification. Examples of tags that can be used include hexahistidine, HA, FLAG, and c-myc epitope. An enzymatic or chemical cleavage site can be engineered between the trefoil peptide and the tag molecule so that the tag can be removed following purification.

Typical expression vectors contain promoters that direct the synthesis of large amounts of mRNA corresponding to the inserted intestinal trefoil peptide nucleic acid in the plasmid-bearing cells. They may also include a eukaryotic or prokaryotic origin of replication sequence allowing for their autonomous replication within the host organism, sequences that encode genetic traits that allow vector-containing cells to be selected for in the presence of otherwise toxic drugs, and sequences that increase the efficiency with which the synthesized mRNA is translated. Stable long-term vectors may be maintained as freely replicating entities by using regulatory elements of, for example, viruses (e.g., the OriP sequences from the Epstein-Barr Virus genome). Cell lines may also be produced that have integrated the vector into the genomic DNA, and in this manner the gene product is produced on a continuous basis. A stable cell line expressing a TDCP or fragment may have a single integrated copy of the vector containing the desired nucleic acid sequences or multiply integrated copies. Nucleic acid sequences encoding trefoil peptides can be amplified in situ by various methods known in the art, for example, by methotrexate selection.

Expression of foreign sequences in bacteria, such as Escherichia coli, requires the insertion of an intestinal trefoil peptide nucleic acid sequence into a bacterial expression vector. Such plasmid vectors contain several elements required for the propagation of the plasmid in bacteria, and for expression of the DNA inserted into the plasmid. Propagation of only plasmid-bearing bacteria is achieved by introducing, into the plasmid, selectable marker-encoding sequences that allow plasmid-bearing bacteria to grow in the presence of otherwise toxic drugs. The plasmid also contains a transcriptional promoter capable of producing large amounts of mRNA from the cloned gene. Such promoters may be (but are not necessarily) inducible promoters that initiate transcription upon induction. The plasmid also preferably contains a polylinker to simplify insertion of the gene in the correct orientation within the vector.

Other bacterial species may also be used to propagate and/or express intestinal trefoil peptides and fragments in a manner similar to using E. coli. For instance, Lactobacilli species may be used to express the TDCPs/fragments either as soluble cytoplasmic proteins or by creating chimeric fusion proteins in which signal peptides would direct the expressed proteins into the periplasmic regions, to the outer surface of the bacteria, or as a secreted product out of the cell. Lactobacilli spp can be further utilized to express foreign proteins in the preparation of consumable food products, for example, in making yogurt or other dairy products.

Mammalian cells can also be used to express a trefoil peptide. Stable or transient cell line clones can be made using intestinal trefoil peptide expression vectors to produce the trefoil peptide in a soluble (truncated and tagged) form. Appropriate cell lines include, for example, COS, HEK293T, CHO, and NIH cell lines such as NIH-3T3.

Once the appropriate expression vectors are constructed, they are introduced into an appropriate host cell by transformation techniques, such as, but not limited to, calcium phosphate transfection, DEAE-dextran transfection, electroporation, bombardment, microinjection, protoplast fusion, dendrimer-mediated transfection, or liposome-mediated transfection. The host cells that are transfected with the vectors of this invention may include (but are not limited to) E. coli or other bacteria, yeast, fungi, insect cells (using, for example, baculoviral vectors for expression in SF9 insect cells), or cells derived from murine, human, or other animals. In vitro expression of trefoil peptides, fusions, or polypeptide fragments encoded by cloned DNA may also be used. Those skilled in the art of molecular biology will understand that a wide variety of expression systems and purification systems may be used to produce recombinant trefoil peptides and fragments thereof, for example, cell-free wheat germ extracts, rabbit reticulocyte extracts, HeLa cell extracts, Kreb's extracts, and E. coli extracts are commonly used in the art for cell-free in vitro translation. Some of these systems are described, for example, in Ausubel et al. (Current Protocols in Molecular Biology, John Wiley & Sons, New York, N.Y. 2000).

Transgenic plants, plant cells, and algae are also particularly useful for generating recombinant TDCPs and trefoil peptide fragments for use in the methods and compositions of the invention. For example, transgenic tobacco plants or cultured transgenic tobacco plant cells expressing a trefoil peptide can be created using techniques known in the art (see, for example, U.S. Pat. Nos. 5,202,422 and 6,140,075). Transgenic algae expression systems can also be used to produce recombinant proteins (see, for example, Chen et al., Curr. Genet. 39:365-370, 2001). Transgenic food-crops, which express recombinant TDCPs or fragments, can be produced with the added benefit that protein isolation would not be necessary. Preferably, the transgenic plants are monocotyledonous, more preferably, the monocots are corn, barley, wheat, oat, rye, rice, and sorghum. Transgenic monocot expression systems are well known in the art (see, for example, U.S. Pat. Nos. 5,850,018, 5,866,793, 5,888,789, 5,889,189, 6,365,807, 6,399,861, and6,403,862; herein incorporated by reference).

In a preferred embodiment, the TDCPs and fragments are expressed in yeast; preferably in Saccaromyces cereviseae, Schizosaccaromyces pombe, or Pichia pastoris. More preferably, expression is facilitated in the methylotrophic yeast, Pichia pastoris expression system. Pichia pastoris is capable of utilizing methanol as a carbon source. Typically, TDCP/fragment nucleic acid sequences are introduced into a Pichia expression vector comprising the 5′ and 3′ promoter and regulatory sequences of the methanol inducible, alcohol oxidase (AOXI) gene, which provides for both targeted integration into the Pichia genome, and for high level of methanol-induced protein production. Polypeptides can be expressed as soluble cytoplasmic proteins or preferably fused in-frame with a secretory signal peptide and expressed as a secreted recombinant polypeptide. Preferably, the secretory signal is based on either the A or α-factor secretory signal. Following methanol induction, recombinant chimeric proteins bearing the A or α-factor secretory signal will be exported out of the cell and can be collected from the media for further purification (see, for example, U.S. Pat. Nos. 4,808,537, 4,837,148, 4,879,231, 4,882,279, 4,818,700, 4,895,800, and 4,812,405, 5,032,516, 5,122,465, 5,268,273; hereby incorporated by reference).

Identification of TDCPs in Yeast Expression Systems

We have observed that production of trefoil peptides in Pichia pastoris leads to post-translational events which include proteolytic cleavage and dipeptide addition. Cleavage of ITF₁₋₇₃ polypeptide fragments has been demonstrated to occur between leu₂₀ and ser₂₁ at the amino terminus and between thr₇₂ and phe₇₃ at the carboxy terminus. Production of this fragment (aa 21-72 of FIG. 1A; hereafter ITF₂₁₋₇₂) has been shown to occur during fermentation and is dependent upon both time and pH. Our studies have shown that longer fermentation times results in increased hITF₂₁₋₇₂ yields. We also observed that the optimal yield of the ITF₂₁₋₇₂ and ITF₂₁₋₇₃ occurs at a pH of ˜5; whereas the optimal yield of the ITF₁₅₋₇₂ and ITF₁₅₋₇₂ occurs at pH 6.0-6.5. As the pI of ITF₁₅₋₇₃ and ITF₂₁₋₇₂ is 5.1 and 6.9 respectively, separation of the two products can be achieved by ion-exchange chromatography.

Alternatively, the production of TDCPs such as hITF₂₅₋₆₂, hITF₂₂₋₆₂, hITF₂₁₋₆₂, hITF₂₅₋₇₃, hITF₂₂₋₇₃, and hITF₂₁₋₇₃ can be attained by fusing the corresponding nucleic acid sequence immediately following an initiator methionine (AUG). Translation of the resulting mRNA in any prokaryotic or eukaryotic host would lead to the cleavage of the initiator methionine by a methionine aminopeptidase (MetAP). MetAPs have been extensively studied and have been shown to cleave the initiator methionine residue if the amino acid at position 2 (i.e., following the methionine) is glycine, alanine, serine, threonine, proline, cysteine, or valine (Arfine et al., Proc. Natl. Acad. Sci. USA, 92:7714-7718, 1995; Bradshaw et al., Trends Biochem. Sci., 23:263-267, 1998; Lowther and Matthews, Biochim. Biophys. Acta, 1477:157-167, 2000).

Dipeptide addition does not significantly affect the biological active of the TDCP. Most commonly, a glutamate-alanine (EA)-N-terminal addition is observed and arises from an alternative processing site in the signal sequence of the Pichia yeast expression system. As described in detail below, an EA-N-terminal addition occurs in the production of ITF₁₅₋₇₃, resulting in a 61 amino acid product (EA-hITF₁₅₋₇₃) which has been detected as a monomer, homomeric dimer, and heteromeric dimer in combination with ITF₁₅₋₇₃.

It is understood that the expression of TDCPs and trefoil peptide fragments in eukaryotic expression systems has the added benefit of being post-translationally processed in the appropriate cellular organelle(s). For instance, glycosylation of TDCPs can be facilitated in the endoplasmic reticulum or golgi apparatus prior to secretion. Secreted proteins can be processed by proteolytic processing by proteases residing at the extracellular face of the cell, such as the proprotein convertases (PCs).

Once a recombinant protein is expressed, it can be isolated from cell lysates if expressed as a cytoplasmic protein, or from the media if expressed as a secreted protein. Protein purification techniques such as ion-exchange, gel-filtration, and affinity chromatography can be utilized to isolate intestinal trefoil peptides from unwanted cellular proteins. Once isolated, the recombinant protein can, if desired, be purified further by high performance liquid chromatography (HPLC; e.g., see Fisher, Laboratory Techniques In Biochemistry And Molecular Biology, Work and Burdon, Eds., Elsevier, 1980).

TDCPs and trefoil peptide fragments of the invention can also be produced by chemical synthesis using, for example, Merrifield solid phase synthesis, solution phase synthesis, or a combination of both (see, for example, the methods described in Solid Phase Peptide Synthesis, 2nd ed., 1984, The Pierce Chemical Co., Rockford, Ill.). Optionally, TDCPs are then be condensed by standard peptide assembly chemistry.

Alternatively, TDCPs and trefoil peptides may be produced in any appropriate manner as a preprodrug, prodrug, or a precursor molecule encompassing a trefoil domain. It is envisioned that such a molecule upon introduction into the body can undergo further processing once inside the host. For example, a TDCP or fragment carrying further amino acid sequences at the amino or carboxy terminus can undergo proteolytic cleavage to produce the active species used for trefoil therapy. This may be facilitated by endogenous proteases, for example, chymotrypsin, pepsin, and trypsin.

Dosages

All of the therapeutic agents employed in the topical compositions of the present invention, including the trefoil component, can be used in the dose ranges currently known and used for these agents. The following are illustrative examples of dose ranges for the active ingredients of the compositions of the invention. Different concentrations of either the TDCP/fragment or the other agents may be employed depending on the clinical condition of the patient, the goal of therapy (treatment or prophylaxis), and anticipated duration or severity of the damage for which the agent is being given. Additional considerations in dose selection include: disease etiology, patient age (pediatric, adult, geriatric), general health and co-morbidity.

The following examples are intended to illustrate the principle of the present invention and circumstances when trefoil peptide therapy is indicated. The following examples are not intended to be limiting.

### EXAMPLE 1

### Recombinant hITF Fragment Production Using Yeast

Frozen cultures of Pichia pastoris (CCM280), encoding a stably integrated recombinant hITF fragment (hITF₁₅₋₇₃), fused to an α-factor secretory signal and regulated by the AOX-1 methanol-responsive promoter, were streaked onto YPD agar plates and grown for two days at 30° C. Individual colonies were used as an inoculum for growth into 10 mL of YPD liquid medium and further grown in a shaking 30° C. incubator. Preparation and growth are directed as follows.

Media Formulation and Growth of Recombinant Yeast

Preparation of 1 L of Basal salts/Trace salts including 16 g/L glycerol (w/v) was performed in a Fembach flask (Fisher Scientific) and inoculated with 10 mL of YPD culture. Basal salt solution (2 ml/L; with or without glycerol) was added before inoculating with the yeast. This mixture was incubated for 2 days with vigorous shaking at 30° C. After two days, the liquid culture was removed and centrifuged for 10 minutes at 9000 rpm. The supernatant was discarded and the pellet resuspended in ⅕ volume of Basal salts/Trace salts+1.5% (w/v) methanol. No glycerol was added in this step. The Fembach flask was covered with cheesecloth and incubated for 48 hours at 30° C. with vigorous shaking.

At the end of 48 hours, the liquid culture was removed and centrifuged for 15 min at 9000 rpm. The medium contained about 5 mg/L of ITF₁₅₋₇₃.

The medium was extensively dialyzed against double distilled water with several changes. The dialysate was then lyophilized and stored at 4° C. for future use. The lyophilized ITF₁₅₋₇₃ from 40-60 liters of culture was collected, resuspended in 50-100 mL of 20 mM formate buffer pH 4.0, and dialyzed against formate buffer (20 mM pH 4.0; 2 changes).

Basal Salts

| | | 1 Liter | | Supplier |
|---|---|---|---|---|
| 1 | Calcium sulfate 2H₂0 | 0.93 | g | Sigma C-7411 |
| 2 | Potassium sulfate | 18.2 | g | Sigma P-4167 |
| 3 | Magnesium sulfate 7H₂0 | 14.9 | g | Sigma M7774 |
| 4 | Potassium hydroxide | 4.13 | g | Fisher P250-1 |
| 5 | Phosphoric acid (85%) | 26.7 | mL | Fisher A242-212 |
| 6 | Ammonium hydroxide | 23.7 | mL | Fisher A6695-212 |

Trace Salts

| Salt | /Liter | |
|---|---|---|
| Cupric sulfate 5H₂0 | 2.0 | g |
| Sodium Iodide | 0.08 | g |
| Manganese sulfate H₂0 | 3.0 | g |
| Sodium molybdate H₂0 | 0.2 | g |
| Boric acid | 0.02 | g |
| Cobalt chloride | 0.5 | g |
| Zinc chloride | 7.0 | g |
| Ferrous sulfate 7H₂0 | 22.0 | g |
| Biotin | 0.2 | g |
| Sulfuric acid (concentrated) | 1.0 | mL |

Basal salts are prepared with vigorous stirring, followed by autoclave sterilization. Glycerol, when present, is added to the basal salt solution at 16 g/L. When glycerol is omitted, the volume is made up by adding 5 mL of ammonium hydroxide.

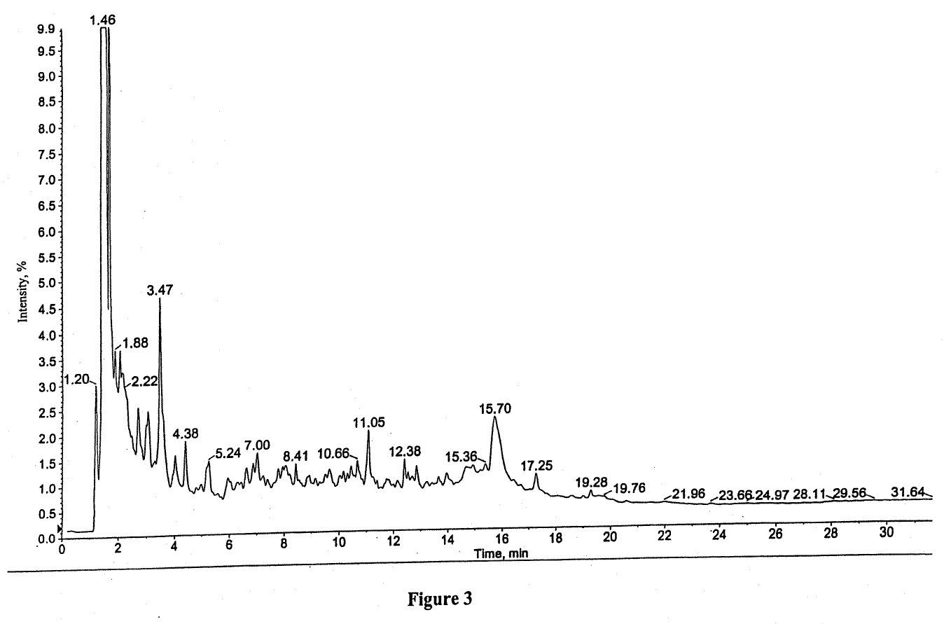

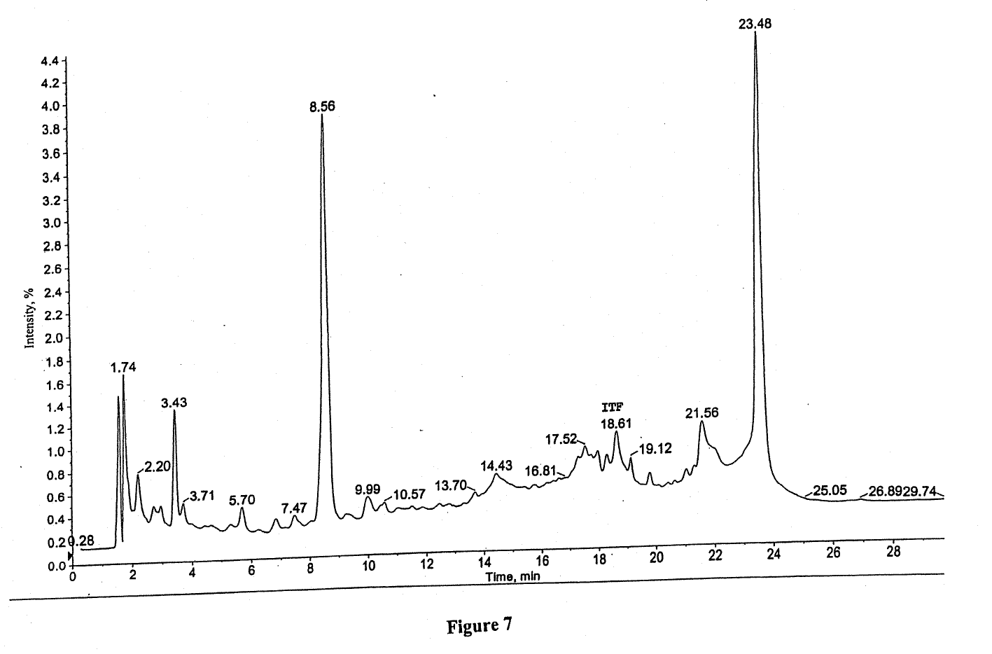

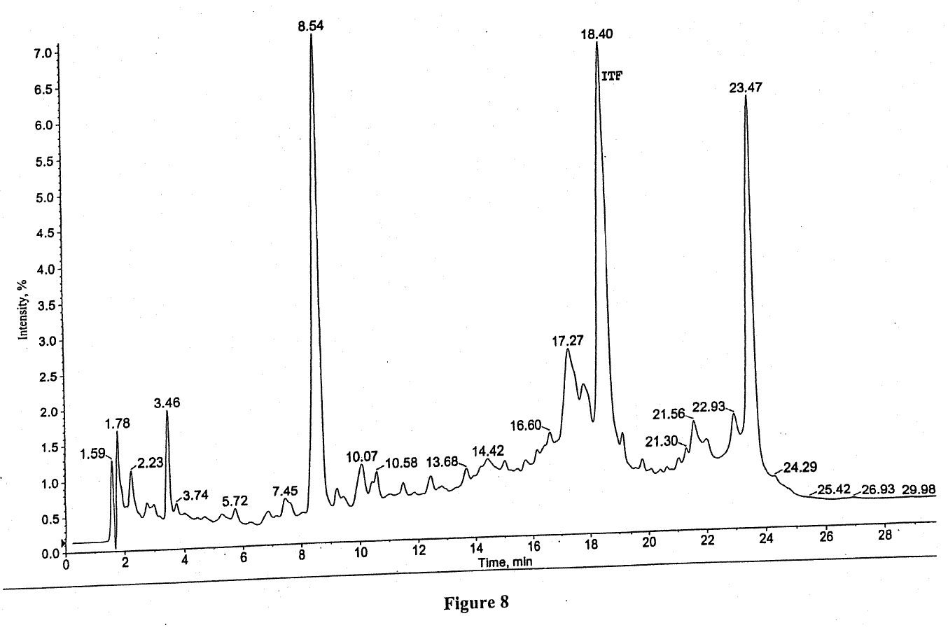

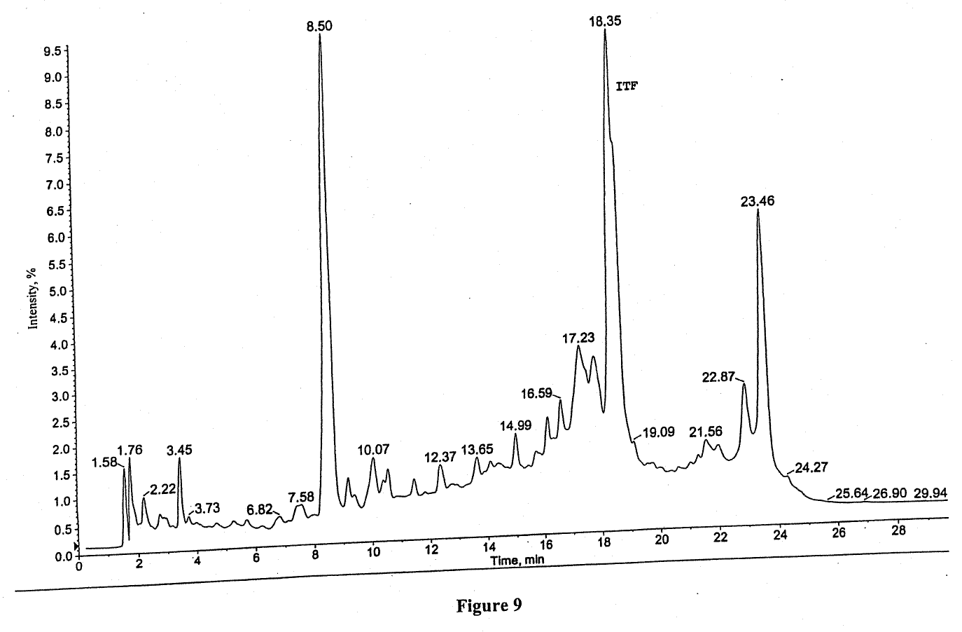

P. pastoris cultures were grown in basal salt media, supplemented with orthophosphate (OP) or hexametaphosphate (HMP) buffer to attain the appropriate pH during growth. The following pH and growth conditions specified in Table 1 were analyzed. Exemplary chromatograms are presented in FIGS. 3-10.

TABLE 1
pH and Growth Conditions in Fermentation Broth

| | | | | Approx. | Final |
|---|---|---|---|---|---|
| | | | | Methanol | Methanol |
| | | | Temp | Induction | Feed Rate |
| Run | Medium | pH | (C.) | (g/L) | (g/min) |
| 16 L-1 | OP | 5.0 | 30 | 200 | 8 |
| 16 L-2 | OP | 5.0 | 30 | 150 | 7 |
| 16 L-3 | OP | 5.0 | 30 | 150 | 10.9 |
| 16 L-4 | OP | 5.0 | 30 | 150 | 10.9 |
| 16 L-5 | OP | 5.0 | 30 | 250 | 15 |
| 16 L-6 | OP | 5.0 | 26 | 250 | 10.9 |
| 16 L-7 | HMP | 5.0 | 30 | 250 | 10.9 |
| 16 L-9 | HMP | 6.0 | 30 | 250 | 10.9 |
| 16 L-10 | HMP | 5.5 | 30 | 250 | 10.9 |
| 100 L-1 | OP | 5.0-6.0 | 27-30 | 250 | 10.9 |

Ten milliliters of sample were removed for analysis at approximately T_{0 hrs}, T_{24 hrs}, T_{36 hrs}, T_{48 hrs}, T_{72 hrs},and T_{96 hrs} time-points following induction with methanol. The samples were centrifuged, and the supernatant decanted and then flash frozen for future analysis.

### EXAMPLE 2

### Analysis of Recombinant hITF in Fermentation Broth Sample Preparation

Recombinant hITF₁₅₋₇₃, purified to homogeneity and prepared in 0.1% aqueous trifluoroacetic acid (TFA), was used as a standard for mass-spectrometry analysis.

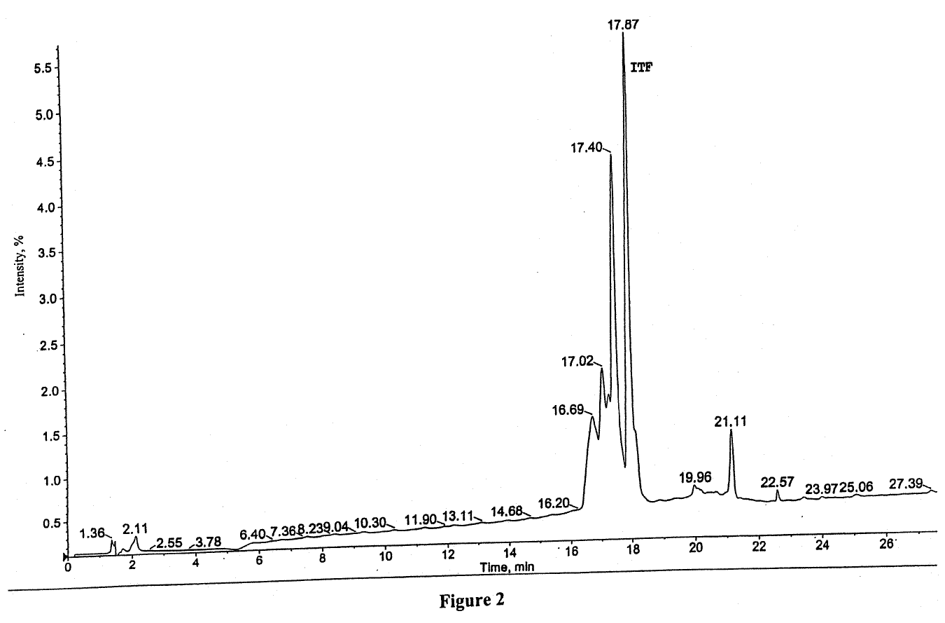

Frozen fermentation broth samples produced in Example 1 were thawed, and 10 μL of 0.1% (v/v) aqueous TFA was added to 90 μL aliquots for analysis. The samples were vortexed and by centrifuged for 5 minutes at 9000 rpm (room temperature). The supernatants were removed and applied to a prewashed (300 μL acetonitrile/0.1% TFA) and equilibrated (500 μL 0.1% TFA) C18 Trap cartridge (Michrom BioResources; Calif., Part No. 004/25109/02). The loaded Trap cartridges were washed with 1 mL 0.1% TFA and bound material was eluted with 100 μL of a 20% acetonitrile/0.1% TFA solution. Samples were dried under N₂ or by lyophilization, resuspended in 50-100 μL of 0.1% TFA, and centrifuged once more for 5 minutes at 9000 rpm. Liquid chromatography-mass spectroscopy (LC-MS) was performed on a 10 μL aliquot of the supernatant (FIG. 2).

Liquid Chromatography-Mass Spectroscopy (LC-MS)

During the course of the investigation, several chromatographic systems were used. The most successful conditions for analysis were as follows:
Solvent A: 0.1% aqueous (v/v) TFA
Solvent B: Acetonitrile containing 0.1% TFA
Gradient: Start at 10% B, hold 2 minutes
   Increase to 40% B over 30 minutes
Flow rate: 0.3 mL/minute
Column: Vydac C18 (2.1×150 mm)
Temperature: 35° C.
Detection: UV₂₁₄ nm
   MS with ESI-MS

Results

LC-MS optimization was performed using the recombinant hITF₁₅₋₇₃ standard described above. The resulting chromatographic profile is provided in FIG. 2 and shows several major components, including those eluting at 17.40 minutes, 17.87 minutes, and 21.1 minutes. When possible, ESI-MS spectra were obtained from the components observed. The following deconvoluted data from the mass spectral profiles are summarized in Table 2 below.

TABLE 2
Summary of LC-MS Results from hITF₁₅₋₇₃ Standard

| Retention Time (min.) | Predicted Mass (Da) |
|---|---|
| 16.69 | 12884 |
| | 9682 |
| 17.02 | 12998 |
| 17.40 | 13470 |
| | 13672 |
| | 13870 |
| 17.87 | 13146 |
| | 13346 |
| 21.11 | 7080 |
| 22.57 | 9706 |

Using the same LC-MS conditions, a sample of fermentation broth (run 16 L-5, pH 5, see Table 1) was analyzed and produced the UV chromatogram shown in FIG. 3. As expected, this shows a complex profile with most analytes eluting between the void region and approximately 22 minutes. Careful examination of the ESI-MS data obtained during the analysis failed to demonstrate the presence of any significant hITF₁₅₋₇₃.

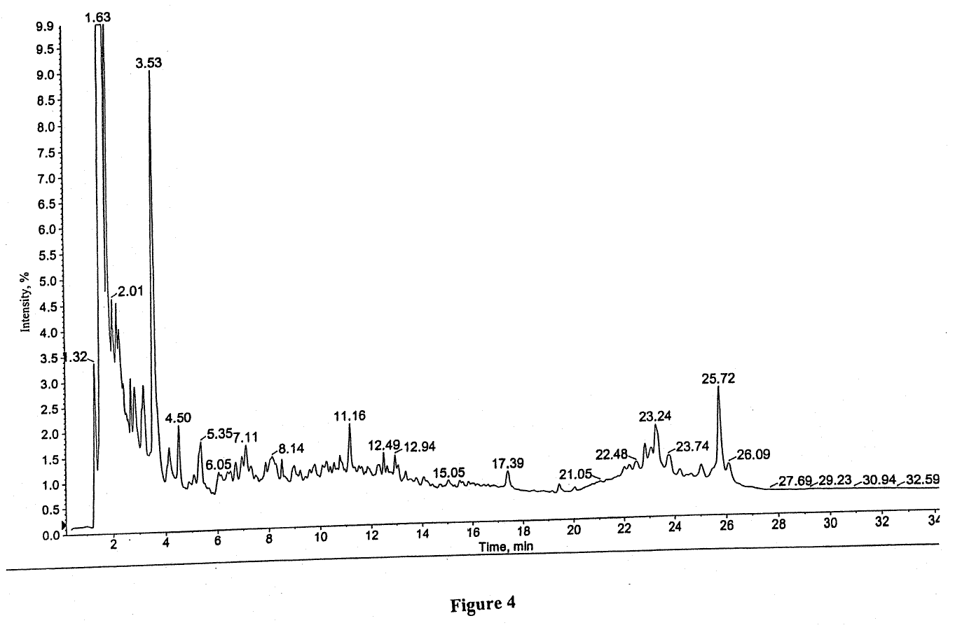

However, during this analysis, a notable protein was detected with a mass of 11764 Da and an elution position during LC-MS of approximately 15.7 minutes (see FIG. 3). The mass, chromatographic behavior, and apparent high quantity suggested this protein may be related to hITF₁₅₋₇₃. Thus, in a further study, the fermentation broth was heated (37° C. for 2 hours) in the presence of a strong reducing agent (DTT) in order to try to remove any disulfide bonds. The resulting chromatogram is provided in FIG. 4. The peak(s) eluting at 15.70 minutes appears to be absent while a number of components, previously undetected are now observed in the 22-26 minute region. Examination of the ESI-MS spectra from these analytes, suggest these to be potential monomers of oxidized proteins. In particular, the peak(s) at 25.7 minutes have masses consistent with monomeric forms of an 11764 Da dimer. Table 3 provides a summary of the MS data obtained from the 22-26 minute region of the chromatogram.

TABLE 3
Summary of LC-MS (22-26 min) from
DTT-Treated Fermentation Broth

| Retention Time (min.) | Predicted Mass (Da) |
|---|---|
| 21.80 | 5535 |
| 22.48 | 5639 |
| | 5741 |
| 22.70 | 5537 |
| 23.05 | 5640 |
| | 5742 |
| 23.24 | 5885 |
| | 6213 |
| | 6633 |
| 25.72 | 5889 |

Again, the atypical behavior of monomer/dimer elution order is characteristic of that observed for hITF₁₅₋₇₃ and indeed the mass of both the monomer and dimer of the major protein species is consistent with an N-terminal truncation of hITF between Leu₂₀ and Ser₂₁ (i.e., hITF₂₁₋₇₃).

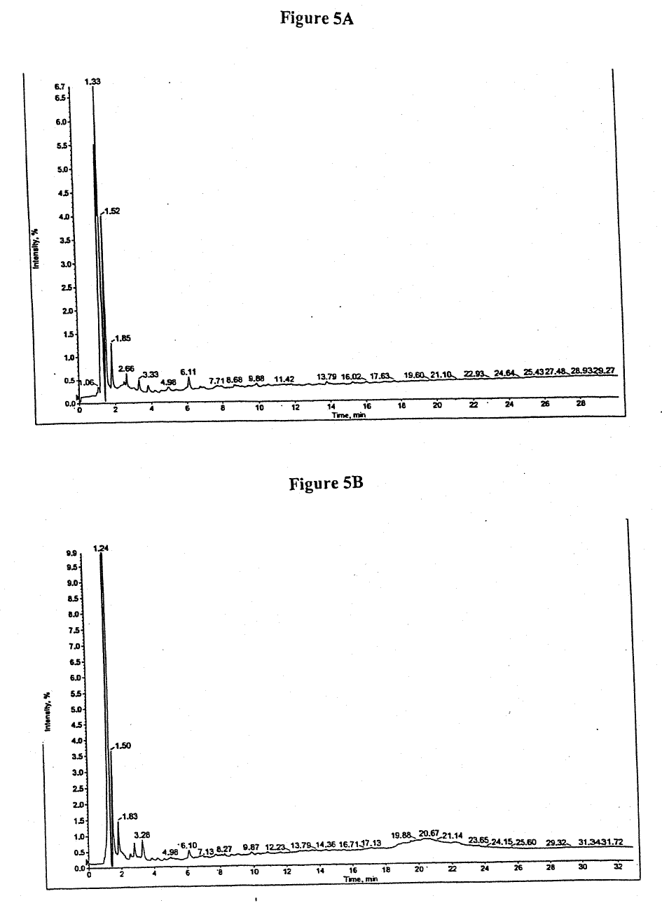

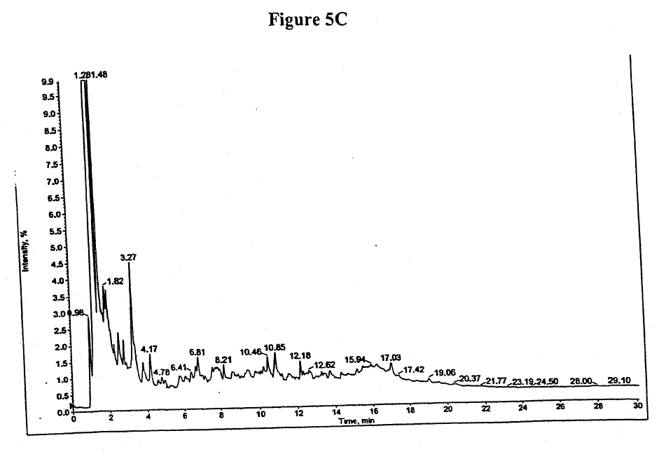

A series of fermentation broth samples (16L-5; see Table 1) were analyzed by LC-MS. As expected, the 0 hour fermentation time produced a relatively “simple” chromatographic profile (FIG. 5A). The complexity of the chromatograms increased as the fermentation progressed as illustrated in FIGS. 5A-5C, which show the results from 0 hours, 22 hours, and 71 hours fermentation times respectively. The ESI-MS data from the 22 hour fermentation sample indicates the presence of some minor products that was not detected in the 0 hour or 91 hour samples. The 91 hour sample did, however, show evidence for the truncated form with mass of 11764 Da.

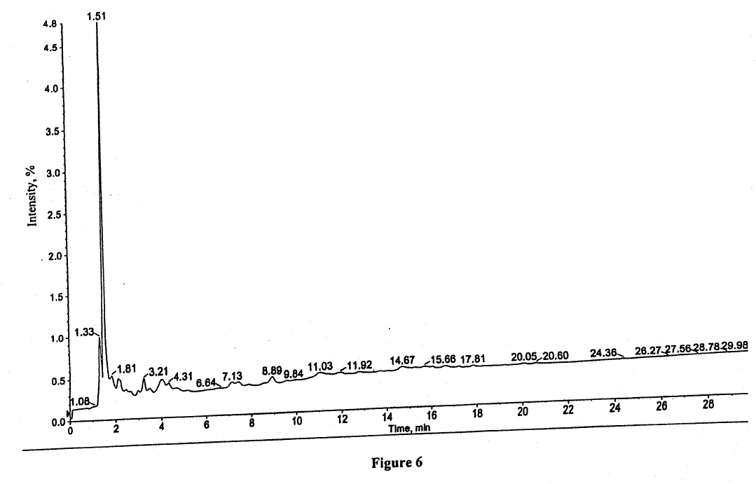

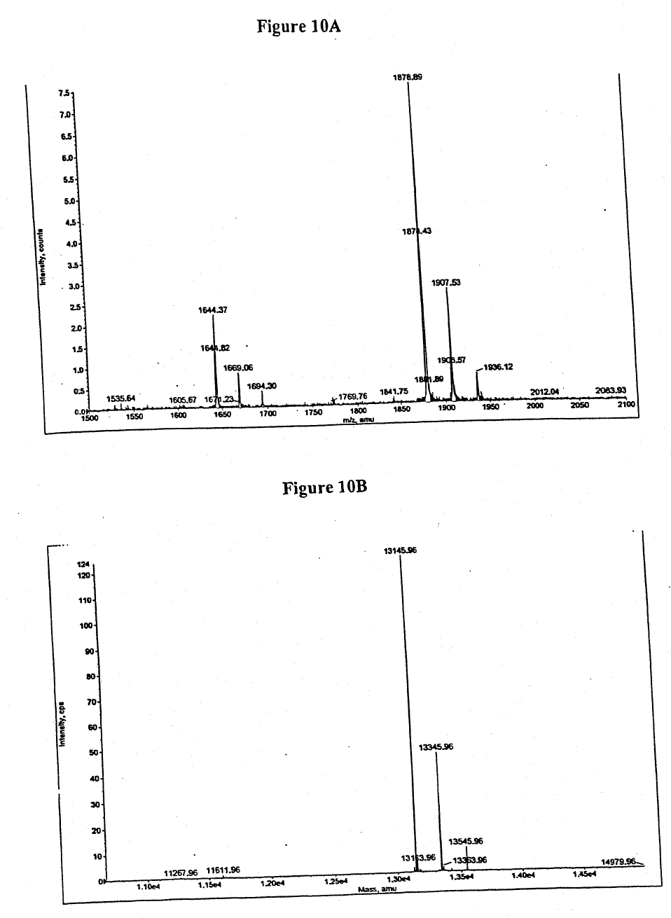

In a final series of experiments, several time points from cultures derived from the 16L-9 (see Table 1) fermentation broth were analyzed by LC-MS. The chromatograms obtained from the 0 hour, 47.5 hour, 72 hour and 96 hour are provided in FIGS. 6-9, respectively. A very notable component is detected at approximately 18.5 minutes, which appears to increase significantly in area as the fermentation progresses. FIGS. 10A and 10B provide the MS data from the 18.5 minute peak and are consistent with that expected for hITF₁₅₋₇₃. No significant “truncated” form of the hITF₁₅₋₇₃ was detected.

Peak area measurements of the 18.5 minute peak were obtained from the chromatograms and these are given in Table 5 together with an estimation of the hITF concentration based upon external reference with a standard of hITF.

Two additional, major UV responsive components are also detected (see FIGS. 6-9). One, eluting at 8.5 minutes gave no assignable MS response. The second, appearing at 23.5 minutes, produced an MS spectrum indicating a mass of 6879 Da.

TABLE 4
Estimated hITF Concentrations in 16L-9 Fermentation Broths

| Fermentation | | Estimated hITF₁₅₋₇₃ |
|---|---|---|
| Time (hours) | Peak Area | Concentration (mg/mL) |
| 0 | ND | — |
| 47.5 | 440 | 0.13 |
| 72 | 11518 | 3.33 |
| 96 | 18930 | 5.48 |
| hITF₁₅₋₇₃ | 3458 | 1.00 |
| Standard | | |

Together, these results demonstrate that the preparation of TDCPs and trefoil peptide fragments, and particularly hITF₁₅₋₇₃ polypeptides (fragments and isoforms), can be influenced according to specific growth conditions. Growth and methanol induction of hITF₁₅₋₇₃-expressing Pichia pastoris at a pH 5 in orthophosphate buffer or pH of 7 and in hexametaphosphate buffer results in the accumulation of truncated hITF polypeptides. In particular, these conditions promote the cleavage of hITF₁₅₋₇₃ between the Leu₂₀ and Ser₂₁ (hITF₂₁₋₇₃). However, the growth of the same yeast at pH 6 in hexametaphosphate or orthophosphate media buffer does not promote this cleavage, resulting in the accumulation of hITF₁₅₋₇₃. If necessary, fragments can be separated from each other by conventional means, such as HPLC, gel filtration, or ion exchange chromatography.

Although the foregoing examples are characterized in terms of Pichia pastoris encoding a stably integrated recombinant hITF₁₅₋₇₃, the yeast strain may be engineered to encode an one or more TDCPs or trefoil peptide fragments. Further, routine optimization of culture conditions, consistent with the principles described here, may be used to maximize the production of the desired TDCP or fragment and its post-translational chemical and enzymatic processing.

### EXAMPLE 3

### The Biologically Activity of TDCPs from Recombinant hITF₁₅₋₇₃

Biological activity of recombinant trefoil peptide fragments was measured using an in vitro wound/migration assay as described by Dignass et al. (J. Clin. Invest. 94:376-383 (1994)). Briefly, primary intestinal epithelial, IEC-6 cells (at passage 17) were grown in sterile Dulbecco's Modified Eagle Medium (DMEM) supplemented with 5% FCS (v/v), 5 μg/μt insulin, and 10 mM penicillin/streptomycin/L-glutamine (PSG). Cells were grown to confluence in a humidified chamber at 37° C. and 5% CO₂.

Prior to performing biopotency assays, the media was removed, washed 2× with 0.1% FCS/DMEM and serum-starved overnight in 0.1% FCS/DMEM at 37° C. and 5% CO₂. Two simulated wounds per well were made by scoring the cells with a razor blade. The cells were then washed once with 0.1% FCS/DMEM. Samples of hITF₁₅₋₇₃ and its fragments and isoforms were produced in Pichia pastoris and purified as described above. The TDCP mixture was then added to separate plates at a concentration of 100 μg per milliliter of 0.1% FCS/DMEM per fragment. A positive control using the known mitogen, transforming growth factor-β (TGF-β), was included at 20 ng/mL media and a negative control of just 0.1% FCS/DMEM were incorporated for later analysis. Cells were incubated for 24 hours at 37° C. At 24 hours post incubation, cells were washed with ice-cold PBS and fixed in cold MeOH. Each plate was then assayed for motility by counting the number of cells crossing the wound edge.

The hITF₁₅₋₇₃ fragments and isoforms (TDCPs) were prepared in the following manner. An SP FastFlow column (Pharmacia) was packed with approximately 200 mL of resin. The resin was prepared with 1 column volume (cv) of 0.5 M NaOH containing 2 M NaCl followed by equilibration with 5 cv of 20 mM formate buffer pH 3.7. The crude TDCP-containing solution was loaded onto the SP FastFlow column (˜2 mL/min). The column was washed with 1 cv of 20 mM formate pH 3.7, then 1 cv of 0.11 M NaCl in 20 mM formate buffer pH 3.7. The TDCPs were eluted using 2 cv of 20 mM sodium citrate pH 6.0. Fractions of pH 4.4-5.8 were collected, corresponding to the OD₂₈₀ rich material. The collected fractions were pooled and dialyzed in 20 mM Tris pH 7.5.

If required, a second chromatographic step was incorporated. A Q-Sepharose column (Pharmacia) was packed with approximately 200 mL of resin. The resin was prepared with 0.2 M HCl containing 5 M NaCl followed by 5 cv of 20 mM Tris pH 7.5. The dialyzed peak from the SP FastFlow column was loaded onto the column containing Q-Sepharose at a flow rate of ˜2 mL/min. The column was washed with 2 column volumes of 20 mM Tris pH 7.5. Elution of hITF₁₅₋₇₃ and its fragments and isoforms was performed with 2 cv of 20 mM Tris pH 7.5 containing 0.1 M NaCl. This will elute mostly monomeric species. Further elutions using 2 cv of 20 mM Tris pH 7.5 containing 0.15 M NaCl promoted the elution of dimerized isoforms.

For performing the biopotency assays, a third chromatographic step was incorporated. The collected fractions obtained from the Q-Sepharose column were then dialyzed extensively in double distilled water (with constant changing). The dialysate was then lyophilized, resuspended in double distilled, deionized HPLC-grade water and purified by reverse-phase HPLC using a YMC phenyl pack 120 A column. Eluted fractions corresponding to major peaks were collected and biopotency was assessed by the in vitro wound healing assay, described above.

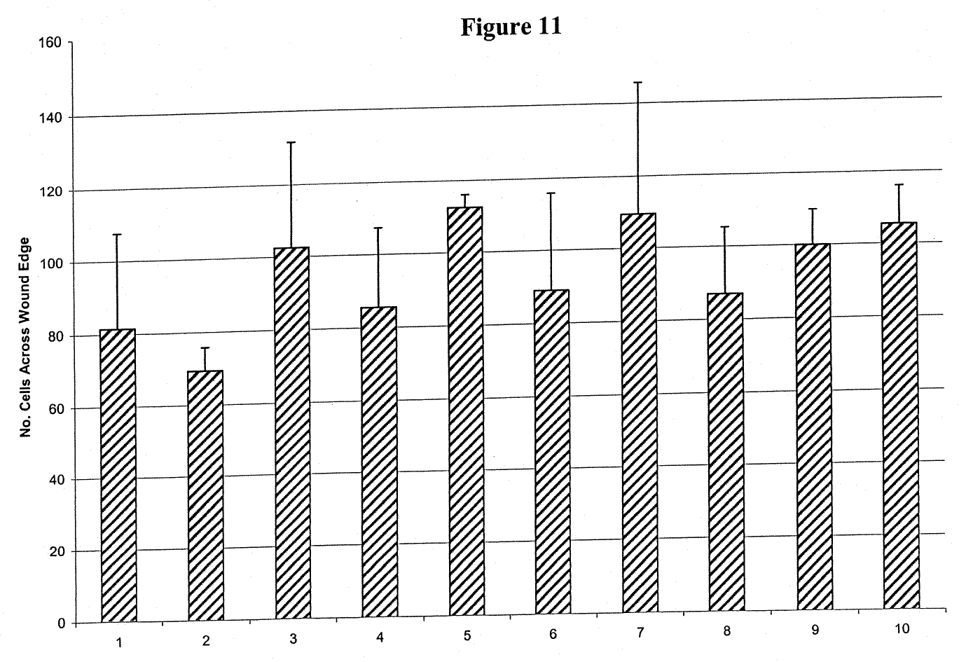

Table 5 summarizes the molecular mass assignments from samples used for the biopotency testing illustrated in FIG. 11. Table 6 summarizes the hITF₁₅₋₇₃ species (i.e., fragments and isoforms) identification based on the molecular mass. In FIG. 11, plate number 1 was treated with 20 ng/mL transforming growth factor-β (TGF-β), a known motility factor. Plate no. 2 shows the effect on IEC-6 cells in the presence of DMEM/0.1% FBS (negative control). Plate no.3 shows the effect of a recombinant purified hITF₁₅₋₇₃ standard at 100 μg/mL (positive control), prepared as described above. Plate nos. 4-10 demonstrates the motility effect of hITF fragments and isoforms (at 100 μg/mL) collected following hITF₁₅₋₇₃ production in Pichia pastoris.

TABLE 5
Collected Samples From hITF-producing Pichia pastoris

| Plate Number | | Retention time of major |
|---|---|---|
| (see FIG. 11) | Molecular Mass (Da) | components (minutes) |
| 4 | 12884 | 6.4 |
| 5 | 12884 | 6.7 |
| 6 | 9682, 13146, 13346 | 9.6, 21.3 |
| 7 | 13146, 13346 | 21.0 |
| 8 | 13470, 13672, 13870 | 19.6 |
| 9 | 13146, 13346 | 21.1 |
| 10 | 13146, 13346 | 21.1 |

TABLE 6
Molecular Mass Identification of hITF₁₅₋₇₃ Species

| Molecular | |
|---|---|
| Mass (Da) | Identification of hITF Fragment |
| 13870 | hITF₁₅₋₇₃ dimer + (Glu, Ala) + (Hexose)₂ |
| 13672 | hITF₁₅₋₇₃ dimer + (Glu, Ala) + (Hexose)₂ |
| 13470 | hITF₁₅₋₇₃ dimer + (Hexose)₂ |
| 13346 | hITF₁₅₋₇₃ dimer + (Glu, Ala) |
| 13146 | hITF₁₅₋₇₃ dimer |
| 12998 | Deletion of one Phe from hITF₁₅₋₇₃ dimer |
| 12884 | i. Deletion of one Phe and one Asn from dimer or |
| | ii. Deletion of two Met residues from dimer |
| 9706 | Unidentified |
| 9682 | Unidentified |
| 7080 | E(1) F(59) - S - S - E(56) F(59) |

Alternative assays to measure biological activity of TDCPs and trefoil peptide fragments can be performed. Such assays are included in, for instance, Taupin et al. (Proc. Natl. Acad. Sci. USA, 97:799-804).

### EXAMPLE 4

### Comparison of the Biologically Activity of Individual TDCPs from Recombinant hITF₁₅₋₇₃

Recombinant hITF₁₅₋₇₃ was produced in Pichia pastoris as described above. The fermentation product was purified by a two step process including Expanded Bed chromatography and hydrophobic interaction chromatography. The recombinant hITF species were purified by ultrafiltration and diafiltration followed by lyophilization. Preliminary analysis confirmed the presence of hITF₁₅₋₇₃ homodimers, EA-hITF₁₅₋₇₃ homodimers, and hITF₁₅₋₇₃/EA-hITF₁₅₋₇₃ heterodimers.

The dimers were purified by reverse phase HPLC and ion exchange chromatography and the identity of each fraction was confirmed by LC-MS (MALDI and Nanospray). N-terminal sequence analysis was also performed for 10 cycles. The dimer fractions were subsequently desalted, lyophilized, and stored at −20° C. for later use. The identification of the hITF dimers are provided in Table 7.

TABLE 7
Characterization of recombinant hITF₁₅₋₇₃ Dimers

| | | IEX Elusion | RP | |
|---|---|---|---|---|
| | | Retention | Retention | Molecular |
| HPLC/IEC | Molecular | Time | Time | Identifi- |
| fraction | Mass (Da) | (minutes) | (minutes) | cation |
| 6 | 13146 | 19.8 | 24.1 | hITF₁₅₋₇₃ |
| | | | | homodimer |
| 8 | 13347 | 25.6 | 24.1 | hITF₁₅₋₇₃/ |
| | | | | EA-hITF₁₅₋₇₃ |
| | | | | heterodimer |
| 11 | 13547 | 31.8 | 24.1 | EA-hITF₁₅₋₇₃ |
| | | | | homodimer |

Biological activity was tested using the in vitro IEC-6 wounding/cell migration assay describe above with the following modifications. IEC-6 cells were cultured to confluence in 24-well multiwell plates and were wounded once with a pipette to yield a 4-5 mm×1 cm wound in each well. Cells were washed and maintained in serum-deprived media, with or without human colonic glycoprotein (hCGP; 2 mg/ml), in the presence of 0.1-1.0 mg/ml of one of the three recombinant hITF dimers identified above for 19 hours. Bovine serum albumin (0.1-1.0 mg/ml) was added instead of an hITF dimer. Wound repair was determined by counting the number of IEC-6 cells across the wound border using a 100 X photomicrograph (inverted Nikon Diaphor TMS, Nikon N6006 camera).

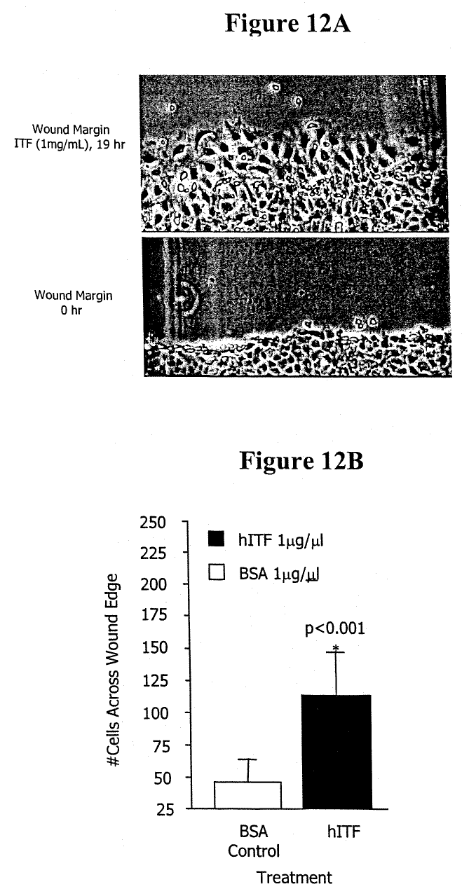

A recombinant hITF₁₅₋₇₃, as prepared in Example 2, produced approximately a 3-fold increase in IEC-6 cells migrating over the wound edge, compared to BSA control (FIG. 12). The relative activity of the hITF₁₅₋₇₃ homodimer, hITF₁₅₋₇₃/EA-hITF₁₅₋₇₃ heterodimer, and EA-hITF₁₅₋₇₃ homodimer was 100%, 95.6%, and 85.3%, respectively.

### Other Embodiments

All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A method of preventing or treating an epithelial lesion in a mammal comprising administering to said mammal a composition comprising a therapeutically effective amount of a trefoil domain-containing polypeptide, or a trefoil peptide fragment, and a mucoadhesive excipient.

2. A method of preventing or treating an eye disorder in a mammal comprising topically administering to the eye of said mammal a composition comprising a therapeutically effective amount of a trefoil domain-containing polypeptide, or a trefoil peptide fragment, and a mucoadhesive excipient.

3. The method of claim 2, wherein said eye disorder affects the cornea, the sclera, the retina, the conjunctiva, the ciliary body, the posterior chamber, or the anterior chamber of the eye.

4. The method of claim 3, wherein said eye disorder affects the corneal epithelium.

5. The method of claim 2, wherein said eye disorder is dry eye.

6. The method of claim 2, wherein said composition comprises eye drops.

7. The method of claim 2, wherein said mucoadhesive comprises a water-soluble polymer.

8. The method of claim 2, wherein said mucoadhesive comprises a polysaccharide.

9. The method of claim 2, wherein said trefoil domain-containing polypeptide or trefoil peptide fragment is administered in a dimeric form.

10. The method of claim 2, wherein said trefoil domain-containing polypeptide or trefoil peptide fragment is encoded by a polynucleotide that hybridizes under high stringency conditions to the coding sequence of human intestinal trefoil factor, human spasmolytic polypeptide, or human pS2.

11. The method of claim 2, wherein said composition further comprises a second therapeutic agent.

12. The method of claim 11, wherein said second therapeutic agent is an anti-inflammatory agent, an antibacterial agent, an antiviral agent, an antifungal agent, an antiprotozoal agent, an analgesic, a steroid, or a 5-aminosalicylate derivative.

13. The method of claim 12, wherein said anti-inflammatory agent is indomethacin, ibuprofen, tacrolimus, acetaminophen, rofecoxib, celecoxib, a salicylic acid derivative, a topical glucocorticoid agent, or a cytokine.

14. The method of claim 12, wherein said antibacterial agent is a penicillin, bacitracin, a cephalosporin, a tetracycline, an aminoglycoside, a macrolide, a fluoroquinolone, chloramphenicol, clindamycin, cycloserine, isoniazid, rifampin, or vancomycin.

15. The method of claim 12, wherein said antiviral agent is 1,-D-ribofuranosyl-1,2,4-triazole-3 carboxamide, 9-2-hydroxy-ethoxy methylguanine, adamantanamine, 5-iodo-2′-deoxyuridine, trifluorothymidine, interferon, adenine arabinoside, a protease inhibitor, a thymidine kinase inhibitor, a sugar or glycoprotein synthesis inhibitor, a structural protein synthesis inhibitor, an attachment or adsorption inhibitor, acyclovir, penciclovir, valacyclovir, or ganciclovir.

16. The method of claim 12, wherein said antifungal agent is Amphotericin B, butylparaben, clindamycin, econaxole, fluconazole, flucytosine, griseofulvin, nystatin, ciclopirox, or ketoconazole.

17. The method of claim 12, wherein said antiprotozoal agent is diloxanide furoate, iodoquinol, paromomycin, dehydroemetine, metronidizole, tinidizole, or ormidazole.

18. The method of claim 12, wherein said analgesic is procaine, lidocaine, tetracaine, dibucaine, benzocaine, p-buthylaminobenzoic acid 2-(diethylamino) ethyl ester HCl, mepivacaine, piperocaine, dyclonine, morphine, codeine, hydrocodone, demorol, or oxycodone.

19. The method of claim 12, wherein said steroid is triamcinolone, hydrocortisone, fluticasone, budesonide, or beclomethasone.

20. The method of claim 12, wherein said 5-aminosalicylate derivative is sulfasalazine, mesalamine, olsalazine, or balsalazide.
